(19) United States
(12) Patent Application Publication
(10) Pub. No.: US 2025/0049346 A1
(43) Pub. Date: Feb. 13, 2025

(54) Automatically Tracking One's Food Consumption Using the "Jedi Mind Trick" Hand Motion

(71) Applicant: Robert A. Connor, Wyoming, MN (US)

(72) Inventor: Robert A. Connor, Wyoming, MN (US)

(73) Assignee: Medibotics LLC, Ham Lake, MN (US)

(21) Appl. No.: 18/929,026

(22) Filed: Oct. 28, 2024

Related U.S. Application Data
(63) Continuation-in-part of application No. 18/885,728, filed on Sep. 15, 2024.
(63) Continuation-in-part of application No. 18/775,128, filed on Jul. 17, 2024.
(63) Continuation-in-part of application No. 18/121,841, filed on Mar. 15, 2023.
Application No. 18/885,728 is a continuation-in-part of application No. 18/775,128, filed on Jul. 17, 2024.
Application No. 18/775,128 is a continuation-in-part of application No. 18/617,950, filed on Mar. 27, 2024.
Application No. 18/775,128 is a continuation-in-part of application No. 18/617,950, filed on Mar. 27, 2024.
Application No. 18/617,950 is a continuation-in-part of application No. 18/121,841, filed on Mar. 15, 2023.
Application No. 18/121,841 is a continuation-in-part of application No. 18/121,841, filed on Mar. 15, 2023.
Application No. 18/121,841 is a continuation-in-part of application No. 17/903,746, filed on Sep. 6, 2022, now abandoned.
Application No. 17/903,746 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 17/239,960, filed on Apr. 26, 2021, now abandoned.
Application No. 17/239,960 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 16/737,052, filed on Jan. 8, 2020, now Pat. No. 11,754,542.
Application No. 16/737,052 is a continuation-in-part of application No. 16/568,580, filed on Sep. 12, 2019, now Pat. No. 11,478,158.
Application No. 16/568,580 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/294,746, filed on Oct. 16, 2016, now Pat. No. 10,627,861.
Application No. 15/294,746 is a continuation-in-part of application No. 15/963,061, filed on Apr. 25, 2018, now Pat. No. 10,772,559.
Application No. 15/963,061 is a continuation-in-part of application No. 15/725,330, filed on Oct. 5, 2017, now Pat. No. 10,607,507.
Application No. 15/725,330 is a continuation-in-part of application No. 15/431,769, filed on Feb. 14, 2017, now abandoned.
Application No. 15/431,769 is a continuation-in-part of application No. 15/418,620, filed on Jan. 27, 2017, now abandoned.
Application No. 15/418,620 is a continuation-in-part of application No. 15/944,746, filed on Apr. 3, 2018, now abandoned.
Application No. 15/963,061 is a continuation-in-part of application No. 14/992,073, filed on Jan. 11, 2016, now abandoned.
Application No. 14/992,073 is a continuation-in-part of application No. 14/550,953, filed on Nov. 22, 2014, now abandoned.
Application No. 14/550,953 is a continuation-in-part of application No. 15/431,769, filed on Feb. 14, 2017, now abandoned.
Application No. 15/431,769 is a continuation-in-part of application No. 14/951,475, filed on Nov. 24, 2015, now Pat. No. 10,314,492.
Application No. 14/951,475 is a continuation-in-part of application No. 15/294,746, filed on Oct. 16, 2016, now Pat. No. 10,627,861.
Application No. 15/294,746 is a continuation-in-part of application No. 15/206,215, filed on Jul. 8, 2016, now abandoned.
Application No. 15/206,215 is a continuation-in-part of application No. 14/992,073, filed on Jan. 11, 2016, now abandoned.
Application No. 14/992,073 is a continuation-in-part of application No. 14/330,649, filed on Jul. 14, 2014, now abandoned.
Application No. 14/330,649 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/951,475, filed on Nov. 24, 2015, now Pat. No. 10,314,492.
Application No. 14/951,475 is a continuation-in-part of application No. 14/948,308, filed on Nov. 21, 2015, now abandoned.
Application No. 14/992,073 is a continuation-in-part of application No. 14/562,719, filed on Dec. 7, 2014, now Pat. No. 10,130,277.
Application No. 14/562,719 is a continuation-in-part of application No. 13/616,238, filed on Sep. 14, 2012, now abandoned.
Application No. 14/951,475 is a continuation-in-part of application No. 14/071,112, filed on Nov. 4, 2013, now abandoned.
Application No. 14/071,112 is a continuation-in-part of application No. 13/901,131, filed on May 23, 2013, now Pat. No. 9,536,449.
Application No. 14/948,308 is a continuation-in-part of application No. 14/550,953, filed on Nov. 22, 2014, now abandoned.
Application No. 14/550,953 is a continuation-in-part of application No. 14/449,387, filed on Aug. 1, 2014, now abandoned.
Application No. 14/449,387 is a continuation-in-part of application No. 14/132,292, filed on Dec. 18, 2013, now Pat. No. 9,442,100.
Application No. 14/132,292 is a continuation-in-part of application No. 13/901,099, filed on May 23, 2013, now Pat. No. 9,254,099.
Application No. 14/330,649 is a continuation-in-part of application No. 13/523,739, filed on Jun. 14, 2012, now Pat. No. 9,042,596.
(60) Provisional application No. 63/542,077, filed on Oct. 2, 2023.
(60) Provisional application No. 63/279,773, filed on Nov. 16, 2021.
(60) Provisional application No. 63/171,838, filed on Apr. 7, 2021.
(60) Provisional application No. 62/930,013, filed on Nov. 4, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/800,478, filed on Feb. 2, 2019.
(60) Provisional application No. 62/857,942, filed on Jun. 6, 2019.
(60) Provisional application No. 62/814,713, filed on Mar. 6, 2019.
(60) Provisional application No. 62/814,692, filed on Mar. 6, 2019.
(60) Provisional application No. 62/549,587, filed on Aug. 24, 2017.
(60) Provisional application No. 62/439,147, filed on Dec. 26, 2016.
(60) Provisional application No. 62/439,147, filed on Dec. 26, 2016.
(60) Provisional application No. 62/349,277, filed on Jun. 13, 2016.
(60) Provisional application No. 62/311,462, filed on Mar. 22, 2016.
(60) Provisional application No. 62/297,827, filed on Feb. 20, 2016.
(60) Provisional application No. 62/349,277, filed on Jun. 13, 2016.
(60) Provisional application No. 62/245,311, filed on Oct. 23, 2015.
(60) Provisional application No. 62/349,277, filed on Jun. 13, 2016.
(60) Provisional application No. 61/932,517, filed on Jan. 28, 2014.

(51) Int. Cl. A61B 5/11; A61B 5/00; G16H 20/60

(52) U.S. Cl. CPC A61B 5/1114; A61B 5/681; G16H 20/60

(57) ABSTRACT

A person's food consumption can be automatically tracked by a finger ring or smart watch with a motion sensor and a camera on its ventral portion. The camera is activated to record images when the device is waved over food. The images are analyzed to identify food types and quantities. A system for tracking food consumption can include a finger ring with a camera which is worn on a person's dominant arm and a smart watch worn on the person's non-dominant arm, wherein images recorded by the camera are displayed by the smart watch.

### CROSS-REFERENCE TO RELATED APPLICATIONS

This application is a continuation-in-part of U.S. patent application Ser. No. 18/885,728 filed on 2024 Sep. 15. This application is a continuation-in-part of U.S. patent application Ser. No. 18/775,128 filed on 2024 Jul. 17. This application is a continuation-in-part of U.S. patent application Ser. No. 18/121,841 filed on 2023 Mar. 15.

U.S. patent application Ser. No. 18/885,728 was a continuation-in-part of U.S. patent application Ser. No. 18/775,128 filed on 2024 Jul. 17. U.S. patent application Ser. No. 18/885,728 was a continuation-in-part of U.S. patent application Ser. No. 18/617,950 filed on 2024 Mar. 27. U.S. patent application Ser. No. 18/885,728 claimed the priority benefit of U.S. provisional application 63/542,077 filed on 2023 Oct. 2. U.S. patent application Ser. No. 18/885,728 was a continuation-in-part of U.S. patent application Ser. No. 18/121,841 filed on 2023 Mar. 15.

U.S. patent application Ser. No. 18/775,128 was a continuation-in-part of U.S. patent application 18/617,950 filed on 2024 Mar. 27. U.S. patent application Ser. No. 18/775,128 was a continuation-in-part of U.S. patent application Ser. No. 18/121,841 filed on 2023 Mar. 15.

U.S. patent application Ser. No. 18/617,950 claimed the priority benefit of U.S. provisional application 63/542,077 filed on 2023 Oct. 2. U.S. patent application Ser. No. 18/617,950 was a continuation-in-part of U.S. patent application Ser. No. 18/121,841 filed on 2023 Mar. 15.

U.S. patent application Ser. No. 18/121,841 was a continuation-in-part of U.S. patent application Ser. No. 17/903,746 filed on 2022 Sep. 6. U.S. patent application Ser. No. 18/121,841 was a continuation-in-part of U.S. patent application Ser. No. 17/239,960 filed on 2021 Apr. 26. U.S. patent application Ser. No. 18/121,841 was a continuation-in-part of U.S. patent application Ser. No. 16/737,052 filed on 2020 Jan. 8.

U.S. patent application Ser. No. 17/903,746 was a continuation-in-part of U.S. patent application Ser. No. 16/568,580 filed on 2019 Sep. 12. U.S. patent application Ser. No. 17/903,746 was a continuation-in-part of U.S. patent application Ser. No. 16/737,052 filed on 2020 Jan. 8. U.S. patent application Ser. No. 17/903,746 was a continuation-in-part of U.S. patent application Ser. No. 17/239,960 filed on 2021 Apr. 26. U.S. patent application Ser. No. 17/903,746 claimed the priority benefit of U.S. provisional application 63/279,773 filed on 2021 Nov. 16.

U.S. patent application Ser. No. 17/239,960 claimed the priority benefit of U.S. provisional application 63/171,838 filed on 2021 Apr. 7. U.S. patent application Ser. No. 17/239,960 was a continuation-in-part of U.S. patent application Ser. No. 16/737,052 filed on 2020 Jan. 8.

U.S. patent application Ser. No. 16/737,052 claimed the priority benefit of U.S. provisional application 62/930,013 filed on 2019 Nov. 4. U.S. patent application Ser. No. 16/737,052 claimed the priority benefit of U.S. provisional application 62/857,942 filed on 2019 Jun. 6. U.S. patent application Ser. No. 16/737,052 claimed the priority benefit of U.S. provisional application 62/814,713 filed on 2019 Mar. 6. U.S. patent application Ser. No. 16/737,052 claimed the priority benefit of U.S. provisional application 62/814,692 filed on 2019 Mar. 6. U.S. patent application Ser. No. 16/737,052 claimed the priority benefit of U.S. provisional application 62/800,478 filed on 2019 Feb. 2. U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/737,052 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/737,052 was a continuation-in-part of U.S. patent application Ser. No. 15/431,769 filed on 2017 Feb. 14. U.S. patent application Ser. No. 16/737,052 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 16/568,580 claimed the priority benefit of U.S. provisional application 62/857,942 filed on 2019 Jun. 6. U.S. patent application Ser. No. 16/568,580 claimed the priority benefit of U.S. provisional application 62/814,713 filed on 2019 Mar. 6. U.S. patent application Ser. No. 16/568,580 claimed the priority benefit of U.S. provisional application 62/814,692 filed on 2019 Mar. 6. U.S. patent application Ser. No. 16/568,580 was a continuation-in-part of U.S. patent application Ser. No. 15/963,061 filed on 2018 Apr. 25 which issued as U.S. Pat. No. 10,772,559 on 2020 Sep. 15. U.S. patent application Ser. No. 16/568,580 was a continuation-in-part of U.S. patent application Ser. No. 15/725,330 filed on 2017 Oct. 5 which issued as U.S. Pat. No. 10,607,507 on 2020 Mar. 31. U.S. patent application Ser. No. 16/568,580 was a continuation-in-part of U.S. patent application Ser. No. 15/431,769 filed on 2017 Feb. 14. U.S. patent application Ser. No. 16/568,580 was a continuation-in-part of U.S. patent application Ser. No. 15/418,620 filed on 2017 Jan. 27. U.S. patent application Ser. No. 16/568,580 was a continuation-in-part of U.S. patent application Ser. No. 15/294,746 filed on 2016 Oct. 16 which issued as U.S. Pat. No. 10,627,861 on 2020 Apr. 21.

U.S. patent application Ser. No. 15/963,061 was a continuation-in-part of U.S. patent application Ser. No. 14/992,073 filed on 2016 Jan. 11. U.S. patent application Ser. No. 15/963,061 was a continuation-in-part of U.S. patent application Ser. No. 14/550,953 filed on 2014 Nov. 22.

U.S. patent application Ser. No. 15/725,330 claimed the priority benefit of U.S. provisional application 62/549,587 filed on 2017 Aug. 24. U.S. patent application Ser. No. 15/725,330 claimed the priority benefit of U.S. provisional application 62/439,147 filed on 2016 Dec. 26. U.S. patent application Ser. No. 15/725,330 was a continuation-in-part of U.S. patent application Ser. No. 15/431,769 filed on 2017 Feb. 14. U.S. patent application Ser. No. 15/725,330 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/431,769 claimed the priority benefit of U.S. provisional application 62/439,147 filed on 2016 Dec. 26. U.S. patent application Ser. No. 15/431,769 claimed the priority benefit of U.S. provisional application 62/349,277 filed on 2016 Jun. 13. U.S. patent application Ser. No. 15/431,769 claimed the priority benefit of U.S. provisional application 62/311,462 filed on 2016 Mar. 22. U.S. patent application Ser. No. 15/431,769 was a continuation-in-part of U.S. patent application Ser. No. 15/294,746 filed on 2016 Oct. 16 which issued as U.S. Pat. No. 10,627,861 on 2020 Apr. 21. U.S. patent application Ser. No. 15/431,769 was a continuation-in-part of U.S. patent application Ser. No. 15/206,215 filed on 2016 Jul. 8. U.S. patent application Ser. No. 15/431,769 was a continuation-in-part of U.S. patent application Ser. No. 14/992,073 filed on 2016 Jan. 11. U.S. patent application Ser. No. 15/431,769 was a continuation-in-part of U.S. patent application Ser. No. 14/330,649 filed on 2014 Jul. 14.

U.S. patent application Ser. No. 15/418,620 claimed the priority benefit of U.S. provisional application 62/297,827 filed on 2016 Feb. 20. U.S. patent application Ser. No. 15/418,620 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/294,746 claimed the priority benefit of U.S. provisional application 62/349,277 filed on 2016 Jun. 13. U.S. patent application Ser. No. 15/294,746 claimed the priority benefit of U.S. provisional application 62/245,311 filed on 2015 Oct. 23. U.S. patent application Ser. No. 15/294,746 was a continuation-in-part of U.S. patent application Ser. No. 14/951,475 filed on 2015 Nov. 24 which issued as U.S. Pat. No. 10,314,492 on 2019 Jun. 11.

U.S. patent application Ser. No. 15/206,215 claimed the priority benefit of U.S. provisional application 62/349,277 filed on 2016 Jun. 13. U.S. patent application Ser. No. 15/206,215 was a continuation-in-part of U.S. patent application Ser. No. 14/951,475 filed on 2015 Nov. 24 which issued as U.S. Pat. No. 10,314,492 on 2019 Jun. 11. U.S. patent application Ser. No. 15/206,215 was a continuation-in-part of U.S. patent application Ser. No. 14/948,308 filed on 2015 Nov. 21.

U.S. patent application Ser. No. 14/992,073 was a continuation-in-part of U.S. patent application Ser. No. 14/562,719 filed on 2014 Dec. 7 which issued as U.S. Pat. No. 10,130,277 on 2018 Nov. 20. U.S. patent application Ser. No. 14/992,073 was a continuation-in-part of U.S. patent application Ser. No. 13/616,238 filed on 2012 Sep. 14.

U.S. patent application Ser. No. 14/951,475 was a continuation-in-part of U.S. patent application Ser. No. 14/071,112 filed on 2013 Nov. 4. U.S. patent application Ser. No. 14/951,475 was a continuation-in-part of U.S. patent application Ser. No. 13/901,131 filed on 2013 May 23 which issued as U.S. Pat. No. 9,536,449 on 2017 Jan. 3.

U.S. patent application Ser. No. 14/948,308 was a continuation-in-part of U.S. patent application Ser. No. 14/550,953 filed on 2014 Nov. 22. U.S. patent application Ser. No. 14/948,308 was a continuation-in-part of U.S. patent application Ser. No. 14/449,387 filed on 2014 Aug. 1. U.S. patent application Ser. No. 14/948,308 was a continuation-in-part of U.S. patent application Ser. No. 14/132,292 filed on 2013 Dec. 18 which issued as U.S. Pat. No. 9,442,100 on 2016 Sep. 13. U.S. patent application Ser. No. 14/948,308 was a continuation-in-part of U.S. patent application Ser. No. 13/901,099 filed on 2013 May 23 which issued as U.S. Pat. No. 9,254,099 on 2016 Feb. 9.

U.S. patent application Ser. No. 14/562,719 claimed the priority benefit of U.S. provisional application 61/932,517 filed on 2014 Jan. 28.

U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26.

The entire contents of these applications are incorporated herein by reference.

### FEDERALLY SPONSORED RESEARCH: Not Applicable

### SEQUENCE LISTING OR PROGRAM: Not Applicable

### BACKGROUND

### Field of Invention

This invention relates to wearable devices for tracking food consumption.

### INTRODUCTION

Many health problems are caused by poor nutrition, including consumption of too much unhealthy food. There are complex behavioral reasons for poor eating habits. Although not a panacea, real time monitoring and modification of a person's food consumption (e.g. to reduce consumption of unhealthy amounts and/or types of food) can help the person to improve their eating habits and health.

### REVIEW OF THE RELEVANT ART

U.S. patent applications 20090012433 (Fernstrom et al., Jan. 8, 2009, “Method, Apparatus and System for Food Intake and Physical Activity Assessment”), 20130267794 (Fernstrom et al., Oct. 10, 2013, “Method, Apparatus and System for Food Intake and Physical Activity Assessment”), and 20180348187 (Fernstrom et al., Dec. 6, 2018, “Method, Apparatus and System for Food Intake and Physical Activity Assessment”), as well as U.S. Pat. No. 9,198,621 (Fernstrom et al., Dec. 1, 2015, “Method, Apparatus and System for Food Intake and Physical Activity Assessment”) and U.S. Pat. No. 10,006,896 (Fernstrom et al., Jun. 26, 2018, “Method, Apparatus and System for Food Intake and Physical Activity Assessment”), disclose wearable buttons and necklaces for monitoring eating with cameras.

U.S. patent application 20150220109 (von Badinski et al., Aug. 6, 2015, “Wearable Computing Device”) and U.S. Pat. No. 9,582,034 (von Badinski et al., Feb. 28, 2017, “Wearable Computing Device”) disclose a finger ring comprising an interior wall, an exterior wall, a flexible circuit board, and a window that facilitates data transmission, battery recharge, and/or status indication. U.S. Pat. No. 9,146,147 (Bakhsh, Sep. 29, 2015, “Dynamic Nutrition Tracking Utensils”) discloses nutritional intake tracking using a smart utensil. U.S. patent application 20150294450 (Eyring, Oct. 15, 2015, “Systems and Methods for Measuring Calorie Intake”) discloses an image-based system for measuring caloric input. U.S. patent application 20150302160 (Muthukumar et al., Oct. 22, 2015, “Method and Apparatus for Monitoring Diet and Activity”) discloses a method and device for analyzing food with a camera and a spectroscopic sensor.

U.S. patent application 20150325142 (Ghalavand, Nov. 12, 2015, “Calorie Balance System”) discloses a calorie balance system with smart utensils and/or food scales. U.S. patent application 20170193854 (Yuan et al., Jan. 5, 2016, “Smart Wearable Device and Health Monitoring Method”) discloses a wearable device with a camera to monitor eating. U.S. patent application 20160073953 (Sazonov et al., Mar. 17, 2016, “Food Intake Monitor”) discloses monitoring food consumption using a wearable device with a jaw motion sensor and a hand gesture sensor. U.S. patent application 20160077587 (Kienzle et al., Mar. 17, 2016, “Smart Ring”) discloses a smart ring with at least one flexion sensor which detects the distance between the sensor and a finger segment. U.S. patent application 20160091419 (Watson et al., Mar. 31, 2016, “Analyzing and Correlating Spectra, Identifying Samples and Their Ingredients, and Displaying Related Personalized Information”) discloses a spectral analysis method for food analysis.

U.S. Pat. No. 10,058,283 (Zerick et al., Apr. 6, 2016, “Determining Food Identities with Intra-Oral Spectrometer Devices”) discloses an intra-oral device for food analysis. U.S. patent application 20160103910 (Kim et al., Apr. 14, 2016, “System and Method for Food Categorization”) discloses a food categorization engine. U.S. patent application 20160140869 (Kuwahara et al., May 19, 2016, “Food Intake Controlling Devices and Methods”) discloses image-based technologies for controlling food intake. U.S. Pat. No. 9,349,297 (Ortiz et al., May 24, 2016, “System and Method for Nutrition Analysis Using Food Image Recognition”) discloses a system and method for determining the nutritional value of a food item. U.S. patent application 20160148535 (Ashby, May 26, 2016, “Tracking Nutritional Information about Consumed Food”) discloses an eating monitor which monitors swallowing and/or chewing.

U.S. patent application 20160148536 (Ashby, May 26, 2016, “Tracking Nutritional Information about Consumed Food with a Wearable Device”) discloses an cating monitor with a camera. U.S. patent application 20160163037 (Dehais et al., Jun. 9, 2016, “Estimation of Food Volume and Carbs”) discloses an image-based food identification system including a projected light pattern. U.S. Pat. No. 9,364,106 (Ortiz, Jun. 14, 2016, “Apparatus and Method for Identifying, Measuring and Analyzing Food Nutritional Values and Consumer Eating Behaviors”) discloses a food container for determining the nutritional value of a food item. U.S. patent application 20160206251 (Kwon et al., Jul. 21, 2016, “Apparatus for Detecting Bio-Information”) and U.S. Pat. No. 10,349,847 (Kwon et al., Jul. 6, 2019, “Apparatus for Detecting Bio-Information”) disclose an apparatus with a light-emitting diode (LED), a laser diode (LD), and an optical detector.

U.S. patent application 20160292563 (Park, Oct. 6, 2016, “Smart Ring”) discloses systems

and methods for pairing a smart ring with a primary device. U.S. patent applications 20160299061 (Goldring et al., Oct. 13, 2016, “Spectrometry Systems, Methods, and Applications”), 20170160131 (Goldring et al., Jun. 8, 2017, “Spectrometry Systems, Methods, and Applications”), 20180085003 (Goldring et al., Mar. 29, 2018, “Spectrometry Systems, Methods, and Applications”), 20180120155 (Rosen et al., May 3, 2018, “Spectrometry Systems, Methods, and Applications”), and 20180180478 (Goldring et al., Jun. 28, 2018, “Spectrometry Systems, Methods, and Applications”) disclose a handheld spectrometer to measure the spectra of objects. U.S. patent application 20160313241 (Ochi et al., Nov. 27, 2016, “Calorie Measurement Device”) disclose, Mar. 17, 2016, “Food Intake Monitor”) discloses a jaw motion sensor to measure food intake.

U.S. patent application 20160350581 (Manuel et al., Dec. 1, 2016, “Smart Ring with Biometric Sensor”) discloses a ring comprising a ring body and a biometric sensor. U.S. patent application 20170061821 (Choi et al., Mar. 2, 2017. “Systems and Methods for Performing a Food Tracking Service for Tracking Consumption of Food Items”) discloses a food tracking service. U.S. patent application 20170156634 (Li et al., Jun. 8, 2017, “Wearable Device and Method for Monitoring Eating”) and U.S. Pat. No. 10,499,833 (Li et al., Dec. 10, 2019, “Wearable Device and Method for Monitoring Eating”) disclose a wearable device with an acceleration sensor to monitor eating. U.S. patent application 20170209095 (Wagner et al., Jul. 27, 2017, “Optical Physiological Sensor Modules with Reduced Signal Noise”) discloses an optical sensor module with light guides which have outwardly-diverging axial directions.

U.S. patent application 20170235933 (von Badinski et al., Aug. 17, 2017, “Wearable Computing Device”) and U.S. Pat. No. 10,156,867 (von Badinski et al., Dec. 18, 2018, “Wearable Computing Device”) disclose a method for using a finger ring to identify an authorized user by illuminating a portion of the user's skin, imaging the portion, and then generating a capillary map. U.S. patent application 20170249445 (Devries et al., Aug. 31, 2017, “Portable Devices and Methods for Measuring Nutritional Intake”) discloses a nutritional intake monitoring system with biosensors. U.S. patent applications 20170292908 (Wilk et al., Oct. 12, 2017, “Spectrometry System Applications”) and 20180143073 (Goldring et al., May 24, 2018, “Spectrometry System Applications”) disclose a spectrometer system to determine spectra of an object.

U.S. patent application 20180005545 (Pathak et al., Jan. 4, 2018, “Assessment of Nutrition Intake Using a Handheld Tool”) discloses a smart food utensil for measuring food mass. U.S. patent application 20180123629 (Wetzig, May 3, 2018, “Smart-Ring Methods and Systems”) discloses a computerized smart ring which is embedded with electronics, software, sensors wherein the ring can be electronically connected to another computing system. U.S. patent application 20180136042 (Goldring et al., May 17, 2018, “Spectrometry System with Visible Aiming Beam”) discloses a handheld spectrometer with a visible aiming beam. U.S. patent application 20180214077 (Dunki-Jacobs, Aug. 2, 2018, “Meal Detection Devices and Methods”) and U.S. Pat. No. 10,791,988 (Dunki-Jacobs, Aug. 2, 2018. “Meal Detection Devices and Methods”) disclose using biometric sensors to detect meal intake and control a therapeutic device.

U.S. patent application 20180242908 (Sazonov et al., Aug. 30, 2018, “Food Intake Monitor”) and U.S. Pat. No. 10,736,566 (Sazonov, Aug. 11, 2020, “Food Intake Monitor”) disclose monitoring food consumption using an ear-worn device or eyeglasses with a pressure sensor and accelerometer. U.S. patent application 20180252580 (Goldring et al., Sep. 6, 2018, “Low-Cost Spectrometry System for End-User Food Analysis”) discloses a compact spectrometer that can be used in mobile devices such as smart phones. U.S. Pat. No. 10,143,420 (Contant, Dec. 4, 2018, “Eating Utensil to Monitor and Regulate Dietary Intake”) discloses a dietary intake regulating device that also monitors physical activity. U.S. patent application 20190013368 (Chung et al., Jan. 10, 2019, “Near-Infrared Light Organic Sensors, Embedded Organic Light Emitting Diode Panels, and Display Devices Including the Same”) discloses an OLED panel which is embedded with a near-infrared organic photosensor. wherein this structure enables biometric recognition.

U.S. patent application 20190025120 (Lee et al., Jan. 24, 2019, “Spectrometer and Spectrum Measurement Method Utilizing Same”) discloses a spectrometer with a first unit spectral filter which absorbs or reflects light in a part of a wavelength band of a light spectrum of an incident target, a second unit spectral filter which absorbs or reflects light in a wavelength band different from the part of the wavelength band, a first light detector configured to detect a first light spectrum passing through the first unit spectral filter, a second light detector configured to detect a second light spectrum passing through the second unit spectral filter, and a processing unit. U.S. patent application 20190033130 (Goldring et al., Jan. 31, 2019, “Spectrometry Systems, Methods, and Applications”) discloses a hand held spectrometer with wavelength multiplexing.

U.S. patent application 20190033132 (Goldring et al., Jan. 31, 2019, “Spectrometry System with Decreased Light Path”) discloses a spectrometer with a plurality of isolated optical channels. U.S. patent application 20190033217 (Kim, Jan. 31, 2019, “Spectrum Measurement Apparatus and Spectrum Measurement Method”) discloses a spectrum measurement apparatus with a plurality of light sources which emit light at different wavelengths, a light detector, and a processor. U.S. patent application 20190041265 (Rosen et al., Feb. 7, 2019, “Spatially Variable Filter Systems and Methods”) discloses a compact spectrometer system with a spatially variable filter. U.S. Pat. No. 10,249,214 (Novotny et al., Apr. 2, 2019, “Personal Wellness Monitoring System”) and U.S. Pat. No. 11,206,980 (Novotny et al., Dec. 28, 2021, “Personal Wellness Monitoring System”) disclose a personal nutrition, health, wellness and fitness monitor that captures 3D images.

U.S. patent application 20190154584 (Ahn et al., May 23, 2019. “Spectroscopy Apparatus, Spectroscopy Method, and Bio-Signal Measuring Apparatus”) discloses a spectroscopy apparatus with a dispersive element which divides an incident light into a plurality of lights having different output angles. U.S. patent application 20190155385 (Lim et al., May 23, 2019, “Smart Ring Providing Multi-Mode Control in a Personal Area Network”) discloses a smart ring which provides multi-mode control in a personal area network. U.S. patent application 20190167190 (Choi et al., Jun. 6, 2019, “Healthcare Apparatus and Operating Method Thereof”) discloses a healthcare apparatus with a plurality of light sources which emit light of different wavelengths, a light detector, and a processor configured to obtain a blood glucose level. U.S. patent application 20190200883 (Moon et al., Jul. 4, 2019, “Bio-Signal Measuring Apparatus and Operating Method Thereof”) discloses a bio-signal measuring apparatus with a photodetector and an array of light sources around the photodetector.

U.S. patent application 20190213416 (Cho et al., Jul. 11, 2019, “Electronic Device and Method for Processing Information Associated with Food”) and U.S. Pat. No. 10,803,315 (Cho et al., Oct. 13, 2020, “Electronic Device and Method for Processing Information Associated with Food”) disclose analysis of food images to obtain nutritional information concerning food items and to recommend food consumption quantities. U.S. Pat. No. 10,359,381 (Lewis et al., Jul. 23, 2019, “Methods and Systems for Determining an Internal Property of a Food Product”) and U.S. Pat. No. 11,313,820 (Lewis et al., Apr. 26, 2022, “Methods and Systems for Determining an Internal Property of a Food Product”) disclose analyzing interior and external properties of food. U.S. patent application 20190236465 (Vleugels, Aug. 1, 2019, “Activation of Ancillary Sensor Systems Based on Triggers from a Wearable Gesture Sensing Device”) discloses an eating monitor with gesture recognition.

U.S. patent application 20190244704 (Kim et al., Aug. 8, 2019, “Dietary Habit Management Apparatus and Method”) discloses a dietary habit management apparatus using biometric measurements. U.S. patent applications 20190244541 (Hadad et al., Aug. 8, 2019, “Systems and Methods for Generating Personalized Nutritional Recommendations”), 20140255882 (Hadad et al., Sep. 11, 2014, “Interactive Engine to Provide Personal Recommendations for Nutrition, to Help the General Public to Live a Balanced Healthier Lifestyle”), and 20190290172 (Hadad et al., Sep. 26, 2019, “Systems and Methods for Food Analysis, Personalized Recommendations, and Health Management”) disclose methods to provide nutrition recommendations based on a person's preferences, habits, medical and activity.

U.S. patent application 20190246977 (Miller et al., Aug. 15, 2019, “Optical Sensor for Wearable Devices”) discloses methods, systems, apparatuses, and/or devices which emit light into a body, receive light from a depth below a surface of the body, and determine a physiological condition of the body. U.S. patent application 20190272845 (Hasan et al., Sep. 5, 2019, “System and Method for Monitoring Dietary Activity”) discloses a system for monitoring dietary activity via a neck-worn device with an audio input unit. U.S. Pat. No. 10,423,045 (Roberts et al., Sep. 24, 2019, “Electro-Optical Diffractive Waveplate Beam Shaping System”) discloses optical beam shaping systems with a diffractive waveplate diffuser. U.S. patent application 20190295440 (Hadad, Sep. 26, 2019, “Systems and Methods for Food Analysis, Personalized Recommendations and Health Management”) discloses a method for developing a food ontology. U.S. Pat. No. 10,444,834 (Vescovi, Oct. 15, 2019, “Devices, Methods, and User Interfaces for a Wearable Electronic Ring Computing Device”) discloses an electronic device with a finger-ring-mounted touchscreen.

U.S. patent application 20190333634 (Vleugels et al., Oct. 31, 2019, “Method and Apparatus for Tracking of Food Intake and Other Behaviors and Providing Relevant Feedback”), 20170220772 (Vleugels et al., Aug. 3, 2017, “Method and Apparatus for Tracking of Food Intake and Other Behaviors and Providing Relevant Feedback”), and 20180300458 (Vleugels et al., Oct. 18, 2018, “Method and Apparatus for Tracking of Food Intake and Other Behaviors and Providing Relevant Feedback”), as well as U.S. Pat. No. 10,102,342 (Vleugels et al., Oct. 16, 2018, “Method and Apparatus for Tracking of Food Intake and Other Behaviors and Providing Relevant Feedback”) and U.S. Pat. No. 10,373,716 (Vleugels et al., Aug. 6, 2019, “Method and Apparatus for Tracking of Food Intake and Other Behaviors and Providing Relevant Feedback”), disclose a method for detecting, identifying, analyzing, quantifying, tracking, processing and/or influencing food consumption.

U.S. Pat. No. 10,739,820 (Wang et al., Aug. 11, 2020, “Expandable Ring Device”) discloses a ring device including force sensors, ultrasonic sensors, inertial measurement units, optical sensors, touch sensors, and other components. U.S. Pat. No. 10,768,666 (von Badinski et al., Sep. 8, 2020, “Wearable Computing Device”) discloses a smart ring which unlocks a client computing device, wherein the ring includes an accelerometer, a gyroscope, and/or other motion sensor. U.S. patent application 20200294645 (Vleugels, Sep. 17, 2020, “Gesture-Based Detection of a Physical Behavior Event Based on Gesture Sensor Data and Supplemental Information from at Least One External Source”) discloses an automated medication dispensing system which recognizes gestures. U.S. Pat. No. 10,790,054 (Vleugels et al., Sep. 29, 2020, “Method and Apparatus for Tracking of Food Intake and Other Behaviors and Providing Relevant Feedback”) discloses a computer-based method of detecting gestures.

U.S. patent applications 20200337635 (Sazonov et al., Oct. 29, 2020, “Food Intake Monitor”) and 20210345959 (Sazonov et al., Nov. 11, 2021, “Food Intake Monitor”) and U.S. Pat. No. 11,006,896 (Sazonov et al., May 18, 2021, “Food Intake Monitor”) and U.S. Pat. No. 11,564,623 (Sazonov et al., Jan. 31, 2023, “Food Intake Monitor”) disclose an optical proximity sensor and/or temporalis muscle activity sensor to monitor chewing. U.S. Pat. No. 10,842,429 (Kinnunen et al., Nov. 24, 2020, “Method and System for Assessing a Readiness Score of a User”) discloses a method and a system for assessing a user's readiness based on their movements. U.S. patent application 20200381101 (Vleugels, Dec. 3, 2020, “Method and Apparatus for Tracking of Food Intake and Other Behaviors and Providing Relevant Feedback”) discloses methods for detecting, identifying, analyzing, quantifying, tracking, processing and/or influencing, related to the intake of food, eating habits, eating patterns, and/or triggers for food intake events, eating habits, or eating patterns.

U.S. patent applications 20200381101 (Vleugels, Dec. 3, 2020, “Method and Apparatus for Tracking of Food Intake and Other Behaviors and Providing Relevant Feedback”) and 20210350920 (Vleugels et al., Nov. 11, 2021, “Method and Apparatus for Tracking of Food Intake and Other Behaviors and Providing Relevant Feedback”) disclose methods for detecting, identifying, analyzing, quantifying, tracking, processing and/or influencing the intake of food, eating habits, eating patterns, and/or triggers for food intake events, eating habits, or eating patterns. U.S. Pat. No. 10,893,833 (Haverinen et al., Jan. 19, 2021, “Wearable Electronic Device and Method for Manufacturing Thereof”) discloses a wearable electronic device made from non-ceramic material. U.S. Pat. No. 10,900,943 (Fernstrom et al, Jan. 26, 2021, “Method, Apparatus and System for Food Intake and Physical Activity Assessment”) discloses monitoring food consumption using a wearable device with two video cameras and an infrared sensor.

U.S. Pat. No. 10,901,509 (Aimone et al., Jan. 26, 2021, “Wearable Computing Apparatus and Method”) discloses a wearable computing device comprising at least one brainwave sensor. U.S. patent application 20210072833 (Mutlu et al., Mar. 11, 2021, “Self-Mixing Interferometry-Based Gesture Input System Including a Wearable or Handheld Device”) discloses a device with one or more SMI sensors which emit beams of electromagnetic radiation, wherein each beam is emitted in a different direction. U.S. Pat. No. 10,952,669 (Shi et al., Mar. 23, 2021. “System for Monitoring Eating Habit Using a Wearable Device”) discloses a wearable device for monitoring cating behavior with an imaging sensor and an electromyography (EMG) sensor. U.S. Pat. No. 10,952,670 (Mori et al., Mar. 23, 2021. “Meal Detection Method, Meal Detection System, and Storage Medium”) discloses meal detection by analyzing arm motion data and heart rate data.

U.S. patent application 20210089126 (Nickerson, Mar. 25, 2021, “Smart Ring”) discloses a smart ring with a capacitive touch sensor. U.S. patent application 20200015697 (Kinreich, Apr. 15, 2021, “Method and System for Analyzing Neural and Muscle Activity in a Subject's Head for the Detection of Mastication”) discloses a wearable apparatus to automatically monitor consumption by analyzing images captured of the environment. U.S. patent application 20210110159 (Shashua et al., Apr. 15, 2021. “Systems and Methods for Monitoring Consumption”) and U.S. Pat. No. 11,462,006 (Shashua et al., Oct. 4, 2022, “Systems and Methods for Monitoring Consumption”) disclose a wearable apparatus to automatically monitor consumption by a user by analyzing images. U.S. Pat. No. 11,013,430 (Tanriover et al., May 25, 2021, “Methods and Apparatus for Identifying Food Chewed and/or Beverage Drank”) discloses methods and apparatuses for identifying food consumption via a chewing analyzer that extracts vibration data.

U.S. patent application 20210183493 (Oh et al., Jun. 17, 2021, “Systems and Methods for Automatic Activity Tracking”) discloses systems and methods for tracking activities (e.g., cating moments) from a plurality of multimodal inputs. U.S. patent application 20210204815 (Koskela et al., Jul. 8, 2021, “An Optical Sensor System of a Wearable Device, A Method for Controlling Operation of an Optical Sensor System and Corresponding Computer Program Product”) discloses a wearable optical sensor system including at least two photo transmitters, a photoreceiver, receiving electronics, and a microcontroller. U.S. patent application 20210289897 (Hsu et al., Sep. 23, 2021, “Smart Ring”) discloses a smart ring with an antenna chip and a metal ring which functions as an antenna.

U.S. patent application 20210307677 (Bi et al., Oct. 7, 2021, “System for Detecting Eating with Sensor Mounted by the Ear”) discloses a wearable device for detecting eating episodes via a contact microphone. U.S. patent application 20210307686 (Catani et al., Oct. 7, 2021, “Methods and Systems to Detect Eating”) discloses methods and systems for automated eating detection comprising a continuous glucose monitor (CGM) and an accelerometer. U.S. Pat. No. 11,188,124 (von Badinski et al., Nov. 30, 2021, “Wearable Computing Device”) discloses a smart ring with a curved housing having a U-shape interior, a motion sensor, a memory, a transceiver, a temperature sensor, and a processor. U.S. Pat. No. 11,188,160 (Liu, Nov. 30, 2021, “Wireless Controlling System Implemented with Smart Ring and Wireless Controlling Method Thereof”) a wireless controlling system including a smart ring and an identification program installed in a mobile device.

U.S. patent application 20210369187 (Raju et al., Dec. 2, 2021, “Non-Contact Chewing Sensor and Portion Estimator”) discloses an optical proximity sensor to monitor chewing. U.S. Pat. No. 11,222,422 (Alshurafa et al., Jan. 11, 2022, “Hyperspectral Imaging Sensor”) discloses a hyperspectral imaging sensor system to identify item composition. U.S. patent application 20220012467 (Kuo et al., Jan. 13, 2022, “Multi-Sensor Analysis of Food”) discloses a method for estimating food composition by 3D imaging and millimeter-wave radar. U.S. Pat. No. 11,275,453 (Tham et al., Mar. 15, 2022, “Smart Ring for Manipulating Virtual Objects Displayed By a Wearable Device”) discloses systems, devices, media, and methods for using a ring to manipulate a virtual object displayed by smart eyewear.

U.S. patent applications 20220085841 (Gretarsson et al., Mar. 17, 2022, “Smart Ring”) and 20220407550 (Gretarsson et al., Dec. 22, 2022, “Smart Ring”) disclose a wearable device which detects inputs, gestures, and/or biometric parameters. U.S. patent application 20220091683 (Beyhs et al., Mar. 24, 2022, “Ring Input Device with Pressure-Sensitive Input”) and U.S. Pat. No. 11,733,790 (Beyhs et al., Aug. 22, 2023, “Ring Input Device with Pressure-Sensitive Input”) disclose a ring with a pressure-sensitive input mechanism. U.S. patent application 20220301683 (Feilner, Sep. 22, 2022, “Detecting and Quantifying a Liquid and/or Food Intake of a User Wearing a Hearing Device”) discloses detecting and quantifying a food intake via a microphone. U.S. patent applications 20220334639 (Sanchez, Oct. 20, 2022, “Projection System for Smart Ring Visual Output”) and U.S. patent application 20220383741 (Sanchez, Dec. 1, 2022, “Non-Visual Outputs for a Smart Ring”), and U.S. Pat. No. 11,462,107 (Sanchez, Oct. 4, 2022, “Light Emitting Diodes and Diode Arrays for Smart Ring Visual Output”), disclose a smart ring system for displaying information concerning driving conditions.

U.S. Pat. No. 11,479,258 (Sanchez, Oct. 25, 2022, “Smart Ring System for Monitoring UVB Exposure Levels and Using Machine Learning Technique to Predict High Risk Driving Behavior”) discloses systems and methods determine a driver's fitness to safely operate a moving vehicle based on UVB exposure. U.S. patent application Ser. No. 11/478,096 (Chung et al., Oct. 25, 2022, “Food Monitoring System”) discloses a serving receptacle, an information processor, and utensils which are used to estimate food quantity. U.S. Pat. No. 11,510,610 (Tanimura et al., Nov. 29, 2022, “Eating Monitoring Method, Program, and Eating Monitoring Device”) discloses an eating monitoring method using a sensor to measure jaw movement. U.S. patent application 20220386885 (Lee et al., Dec. 8, 2022, “Wearable Electronic Device Measuring Blood Pressure and Method for Operating The Same”) discloses a wearable electronic device with a memory, a first sensor, a second sensor, and a processor.

U.S. patent application 20220400195 (Churovich et al., Dec. 15, 2022, “Electronic Visual Food Probe”) and U.S. Pat. No. 11,366,305 (Churovich et al., Jun. 21, 2022, “Electronic Visual Food Probe”) disclose an electronic visual food probe to view inside food. U.S. patent application 20220409072 (Kang et al., Dec. 29, 2022, “Apparatus and Method for Estimating Bio-Information”) discloses an apparatus to estimate biometric parameters using a pulse wave sensor with channels in an isotropic shape. U.S. Pat. No. 11,540,599 (Yokoyama et al., Jan. 3, 2023, “Watch Band with Adjustable Fit”) discloses shape-memory tensioning elements which respond to a stimulus in order to adjust the fit of a watch band. U.S. patent application 20230000405 (Lee et al., Jan. 5, 2023, “Apparatus and Method for Estimating Bio-Information Based on Bio-Impedance”) discloses an apparatus to estimate biometric parameters using an impedance sensor, including a pair of input electrodes and a pair of receiving electrodes.

U.S. patent application 20230007884 (Kang et al., Jan. 12, 2023, “Apparatus and Method for Estimating Bio-Information”) discloses an apparatus to estimate biometric parameters which measures pulse wave signals from an object. U.S. patent application 20230008487 (Caizzone et al., Jan. 12, 2023, “System and Method for Smart Rings Employing Sensor Spatial Diversity”) discloses a ring for photoplethysmographic sensing which uses sensor spatial diversity to enhance the quality and the reliability of measurements. U.S. patent application 20230021838 (Tse et al., Jan. 26, 2023, “Wearable Electronic Device”) discloses a wearable electronic device with conductive areas on both inner and outer surfaces. U.S. Pat. No. 11,568,760 (Meier, Jan. 31, 2023, “Augmented Reality Calorie Counter”) discloses using chewing noises and food images to estimate food volume.

U.S. patent application 20230034807 (McDaniel et al., Feb. 2, 2023, “Systems and Methods Including a Device for Personalized Activity Monitoring Involving the Hands”) discloses a wearable device for activity monitoring involving the use of hands. U.S. patent application 20230043018 (Wai et al., Feb. 9, 2023, “Smart Ring for Use with a User Device and Wi-Fi Network”) discloses a smart ring with a battery, a memory, processing circuitry, a plurality of sensors, and a plurality of antennas. U.S. Pat. No. 11,580,300 (Tham et al., Feb. 14, 2023, “Ring Motion Capture and Message Composition System”) discloses systems, devices, media, and methods for composing and sharing a message based on the motion of a ring. U.S. patent application 20230053252 (Jung, Feb. 16, 2023, “Electronic Device Adjusting Oxygen Saturation and Method for Controlling the Same”) discloses a device with a first sensor which detects movement and a second sensor which measures oxygen saturation.

U.S. patent applications 20230056434 (Jang et al., Feb. 23, 2023, “Apparatus and Method for Estimating Blood Pressure”) and 20230070636 (Kang et al., Mar. 9, 2023, “Apparatus and Method for Estimating Blood Pressure”) disclose an apparatus for estimating blood pressure using a pulse wave sensor. U.S. Pat. No. 11,599,147 (von Badinski et al., Mar. 7, 2023, “Wearable Computing Device”) discloses a smart ring with a curved housing having a U-shape interior, a semi-flexible PCB, a motion sensor, a memory, a transceiver, a temperature sensor, and a processor. U.S. patent application 20230072436 (Sanchez, Mar. 9, 2023, “Harvesting Energy for a Smart Ring Via Piezoelectric Charging”) discloses a smart ring which harvests mechanical energy using piezoelectricity. U.S. patent application 20230085555 (Nomvar et al., Mar. 16, 2023, “A Non-Invasive Continuous Blood Glucose Monitor”) discloses a non-invasive device for measuring glucose levels.

U.S. patent application 20230118067 (Chang et al., Apr. 20, 2023, “Electronic Device and Method to Measure Bioelectrical Impedance”) discloses an electronic device with a plurality of electrodes, a sensor connected to the electrodes, a memory, and a processor which obtains contact impedances through the sensor. U.S. patent applications 20230143293 (Sanchez, May 11, 2023, “Biometric Authentication Using a Smart Ring”) and U.S. patent application 20230153416 (Sanchez, May 18, 2023, “Proximity Authentication Using a Smart Ring”) discloses systems and methods for performing biometric authentication using a smart ring. U.S. patent application 20230154033 (Oh et al., May 18, 2023, “Method and Device for Estimating Poses and Models of Object”) discloses a method for object pose and model estimation via acquiring a global feature of an input image. U.S. patent application 20230157645 (Lee et al., May 25, 2023, “Apparatus and Method for Estimating Bio-Information”) discloses an apparatus to estimate biometric parameters using a spectrometer.

U.S. Pat. No. 11,660,228 (Goff et al., May 30, 2023, “Positional Obstructive Sleep Apnea Detection System”) discloses an obstructive sleep apnea detection device which uses an optical engagement surface adapted to engage a user's skin. U.S. Pat. No. 11,666,230 (Piccinini et al., Jun. 6, 2023, “Electronic Device and Method for Noninvasive, Continuous Blood Pressure Monitoring”) discloses an electronic device and method for continuous noninvasive blood pressure monitoring. U.S. patent application 20230174114 (Sanchez, Jun. 8, 2023, “Smart Ring System for Measuring Stress Levels and Using Machine Learning Techniques to Predict High Risk Driving Behavior”) discloses systems and methods determine a driver's fitness to safely operate a moving vehicle based on their stress level. U.S. patent application 20230178213 (Haertel et al., Jun. 8, 2023, “Automatic Tracking of Probable Consumed Food Items”) discloses a method for detecting information associated with consumed food.

U.S. patent application 20230190118 (Park et al., Jun. 22, 2023, “Apparatus and Method for Estimating Blood Pressure”) discloses an apparatus for estimating blood pressure which extracts a cardiac output feature, a first candidate total peripheral resistance feature, and a second candidate peripheral resistance feature. U.S. patent application 20230200744 (Choi et al., Jun. 29, 2023, “Apparatus and Method for Estimating Target Component”) discloses an apparatus for estimating a target component via a spectrometer. U.S. patent application 20230205170 (Sanchez, Jun. 29, 2023, “Soft Smart Ring and Method of Manufacture”) discloses a smart ring with a body made from flexible material, a first part, a second part removably connected to the first part, and at least one pair of break-away portions disposed within the body.

U.S. patent application 20230205325 (Khan, Jun. 29, 2023, “Wearable Apparatus and Control Method Thereof”) discloses a wearable apparatus with a display, a strap, at least one sensor configured to acquire posture information, and at least one processor. U.S. patent application 20230213970 (von Badinski et al., Jul. 6, 2023, “Wearable Computing Device”) discloses a smart ring with a body having an inner surface and an outer surface, wherein a cavity is formed on the inner surface of the body part and an electronic part is arranged in the cavity. U.S. patent application 20230213970 (von Badinski et al., Jul. 6, 2023, “Wearable Computing Device”) discloses a smart ring with a curved housing having a U-shape interior, a semi-flexible PCB, a motion sensor, a memory, a transceiver, a temperature sensor, and a processor.

U.S. patent application 20230218192 (Eom et al., Jul. 13, 2023, “Wrist-Type Body Component Measuring Apparatus and Body Component Measuring Method Using the Same”) discloses a wrist-worn band with: a first input electrode and a first output electrode disposed on an inside surface of the band; and a second input electrode and a second output electrode disposed on an outside surface of the band. U.S. patent application 20230225671 (Kosman et al., Jul. 20, 2023, “Wearable Health Apparatus for the Collection of Wellness Data and Providing Feedback Therefrom to the Wearer”) discloses a ring with replaceable outer shells, as well as hardware and software that allow the user to communicate with a cell phone, cloud provider, table, personal computer or AI assistant.

U.S. patent application 20230233084 (Moon et al., Jul. 27, 2023, “Method and Apparatus for Correcting Error of Optical Sensor, and Apparatus for Estimating Biometric Information”) discloses a method of correcting an optical sensor error by adjusting the brightness of a light source. U.S. patent application 20230297858 (Vleugels et al., Sep. 21, 2023, “Nutritional Content Determination Based on Gesture Detection Data”) discloses techniques for nutritional content determination based on gestures. U.S. patent application 20230301540 (Jung et al., Sep. 28, 2023, “Apparatus and Method of Measuring Bio Signal”) discloses a method of measuring a biosignal by positioning electrodes and switching an impedance measurer.

U.S. patent applications 20230309844 (Jang et al., Oct. 5, 2023, “Apparatus and Method for Estimating Blood Pressure”) and 20240172945 (Park et al., May 3, 2024, “Apparatus and Method for Estimating Blood Pressure”) disclose an apparatus and method for estimating blood pressure using a photoplethysmogram (PPG) sensor. U.S. patent application 20230324293 (Lee, Oct. 12, 2023, “Apparatus and Method for Estimating Body Water Status”) discloses an apparatus for estimating body hydration level with a near-infrared light spectrometer. U.S. Pat. No. 11,793,454 (Kinnunen et al., Oct. 24, 2023, “Method and System for Providing Feedback to User for Improving Performance Level Management Thereof”) discloses a method of collecting a set of user information and determining a user's current performance.

U.S. patent application 20230350492 (Nickerson, Nov. 2, 2023, “Smart Ring”) discloses a smart ring worn which is controlled based on its position. U.S. patent application 20230350503 (D'Amone et al., Nov. 2, 2023, “Ring Input Devices”) and U.S. Pat. No. 11,714,494 (D'Amone et al., Aug. 1, 2023, “Ring Input Devices”) disclose how a head-mountable device can be operated with a ring input device worn on a finger of a user. U.S. patent application 20230359291 (Beyhs et al., Nov. 9, 2023, “Ring Input Device with Variable Rotational Resistance”) discloses a ring input device with variable rotational resistance mechanisms which change the rotational friction of a rotating outer band. U.S. patent application 20230361588 (Sanchez, Nov. 9, 2023, “Smart Ring Power and Charging”) discloses a smart ring with a both a removable power source and an internal power source.

U.S. patent application 20230376071 (von Badinski et al., Nov. 23, 2023, “Wearable Computing Device”) discloses a smart ring comprising an external housing component with an outer circumferential surface and an inner circumferential surface, wherein a portion of the inner circumferential surface contacts a person's finger. U.S. patent applications 20230376072 (von Badinski et al., Nov. 23, 2023, “Wearable Computing Device”) and 20230384827 (von Badinski et al., Nov. 30, 2023, “Wearable Computing Device”) disclose a smart ring with a curved housing having a U-shape interior, a curved battery, a semi-flexible PCB, a motion sensor, a memory, a transceiver, a temperature sensor, and a processor. U.S. Pat. No. 11,829,831 (Ershov et al., Nov. 28, 2023, “Electronic System with Ring Device”) discloses a wearable electronic device with a coil which is formed from metal traces.

U.S. patent application 20230409080 (von Badinski et al., Dec. 21, 2023, “Wearable Computing Device”) discloses a smart ring with a curved housing with a substantially transparent portion, a curved battery, a semi-flexible PCB, a motion sensor, a memory, a transceiver, a temperature sensor, and a processor. U.S. Pat. No. 11,850,069 (Mars et al., Dec. 26, 2023, “Wearable Device and Methods of Manufacturing”) discloses a smart ring with a battery, a PCB, a fingerprint sensor, a temperature sensor, a memory, and a processing unit. U.S. Pat. No. 11,862,037 (Seymore et al., Jan. 2, 2024, “Methods and Devices for Detection of Eating Behavior”) discloses systems, devices, and methods using audio data to detect and correct eating behavior. U.S. patent application 20240000380 (Fei et al., Jan. 4, 2024, “Wearable Device”) discloses an annular case with an energy storage unit, an information transmission unit, and an optical identification assembly.

U.S. patent application 20240000387 (Realubit et al., Jan. 4, 2024, “Finger Wearable Health Monitoring Device”) discloses a finger-worn health monitoring device comprising a circular metal shell. U.S. Pat. No. 11,864,871 (Kang et al., Jan. 9, 2024, “Wearable Device and Method of Measuring Bio-Signal”) discloses an external light collector, an auxiliary light source, and a light receiver. U.S. Pat. No. 11,868,178 (von Badinski et al., Jan. 9, 2024, “Wearable Computing Device”) discloses a smart ring with a curved housing having a U-shape interior, a battery, a semi-flexible PCB, a motion sensor, a memory, a transceiver, a temperature sensor, and a processor. U.S. Pat. No. 11,868,179 (von Badinski et al., Jan. 9, 2024, “Wearable Computing Device”) discloses a smart ring with a curved housing having a U-shape interior, a battery, a semi-flexible PCB, a motion sensor, a memory, a transceiver, a temperature sensor, a processor, an infrared light emitter, and a visible light emitter.

U.S. patent application 20240012479 (Qiu et al., Jan. 11, 2024, “Ring Enabling Its Wearer to Enter Control Commands”) discloses systems and methods, including a smart ring, which enable a user to control electronic devices in a local network. U.S. Pat. No. 11,874,701 (von Badinski et al., Jan. 16, 2024, “Wearable Computing Device”) discloses a smart ring with a curved housing having a U-shape interior, a battery, a semi-flexible PCB, a motion sensor, a memory, a transceiver, a temperature sensor, and a processor which identifies gestures based on data from the motion sensor. U.S. Pat. No. 11,874,702 (von Badinski et al., Jan. 16, 2024, “Wearable Computing Device”) discloses a smart ring with a curved housing with a substantially transparent portion, a battery, a semi-flexible PCB, a motion sensor, a memory, a transceiver, a temperature sensor, and a processor.

U.S. patent application 20240028294 (Li et al., Jan. 25, 2024, “Automatic Quantitative Food Intake Tracking”) discloses methods and devices for tracking food intake using smart glasses. U.S. patent application 20240029337 (Parker et al., Jan. 25, 2024, “Employing Controlled Illumination for Hyperspectral or Multispectral Imaging of Food in an Appliance”) discloses a method for capturing 2D images of objects illuminated by optical radiation. U.S. Pat. No. 11,895,383 (Prushinskiy et al., Feb. 6, 2024, “Electronic Device Including Optical Sensor”) discloses an electronic device with a housing which is rotatably arranged and an optical sensor assembly. U.S. patent application 20240045473 (Lee et al., Feb. 8, 2024, “Electronic Device and Method for Operating Electronic Device”) discloses an electronic device with movable housings, at least one sensor, and one or more electromagnets.

U.S. patent application 20240046505 (Liu et al., Feb. 8, 2024, “Electronic Device and Method with Pose Prediction”) discloses an electronic device for predicting a pose and a method for operating the electronic device. U.S. patent application 20240048675 (Choi et al., Feb. 8, 2024, “Electronic Device and Operation Method Thereof”) discloses a device with processors which obtain a rotation angle of the device and determine whether the rotation angle is greater than or equal to a reference rotation angle. U.S. patent application 20240049350 (Choi et al., Feb. 8, 2024, “Electronic Apparatus and Operating Method Thereof”) discloses an electronic which can receive a requests for wireless connection from a first device, identify a usage of a wireless bandwidth of the first device, and determine whether a wireless connection to the first device is possible based on a remaining wireless bandwidth of the electronic device.

U.S. Pat. No. 11,902,791 (Mars et al., Feb. 13, 2024, “Reader Device with Sensor Streaming Data and Methods”) discloses an access control system with a controller having an antenna interface to broadcast identifying data. U.S. patent application 20240055101 (Panetta et al., Feb. 15, 2024, “Food and Nutrient Estimation, Dietary Assessment, Evaluation, Prediction and Management”) discloses using artificial intelligence (AI) automatic methods, computer program product, and systems and methodology for dietary and medical treatment planning. U.S. patent application 20240058686(Bhandarkar et al., Feb. 22, 2024, “Smart Wearable Device”) discloses a smart ring with an electronics unit that is selectively attachable to a coupling mount. U.S. Pat. No. 11,911,181 (Huttunen et al., Feb. 27, 2024, “Flexible Wearable Ring Device”) discloses a wearable device made from flexible materials.

U.S. Pat. No. 11,916,900 (Mars et al., Feb. 27, 2024, “Authorized Remote Control Device Gesture Control Methods and Apparatus”) discloses a method for controlling a remote control device which includes capturing biometric data. U.S. patent application 20240065631 (Brooks, Feb. 29, 2024, “Pressure Adjustment for Biometric Measurement”) discloses a user device with a pressure sensor to determine whether the pressure between a user's body and the device is within a proper range. U.S. Pat. No. 11,925,441 (Rantanen et al., Mar. 12, 2024, “Techniques for Determining Blood Pressure Based on Morphological Features of Pulses Preliminary Class”) discloses a wearable device with one or more light emitting components, one or more photodetectors, and a controller that couples the light emitting components to the photodetectors.

U.S. patent application 20240081663 (Park et al., Mar. 14, 2024, “Apparatus for Estimating Bio-Information and Method of Detecting Abnormal Bio-Signal”) discloses an apparatus with a photoplethysmogram (PPG) sensor. U.S. patent application 20240096117 (Li et al., Mar. 21, 2024, “Multi-Spectrum Camera System to Enhance Food Recognition”) discloses a camera system to enhance food and material identification. U.S. Pat. No. 11,937,905 (Singleton et al., Mar. 26, 2024, “Techniques for Leveraging Data Collected by Wearable Devices and Additional Devices”) discloses a method comprising receiving physiological data from a wearable device and environmental data from an external device. U.S. Pat. No. 11,949,673 (Sanchez, Apr. 2, 2024, “Gesture Authentication Using a Smart Ring”) discloses systems and methods for multi-factor authentication using a smart ring.

U.S. patent application 20240112563 (Norman et al., Apr. 4, 2024, “Bluetooth Enabled Smart Ring”) discloses a smart ring device with wireless communication with a computing device that transitions between one or more states based on user and/or device inputs. U.S. patent application 20240115212 (Jang et al., Apr. 11, 2024, “Apparatus and Method for Estimating Physiological Variables”) discloses an apparatus for estimating physiological variables using sensors and a neural-network-based physiological variable estimation model. U.S. patent application 20240125915 (Au et al., Apr. 18, 2024, “Method, Apparatus, and System for Wireless Sensing Measurement and Reporting”) and U.S. patent application 20240179550 (Au et al., May 30, 2024, “Method, Apparatus, and System for Wireless Sensing Measurement and Reporting”) disclose methods, devices, and systems for wireless sensing including transmitting a time series of at least one wireless sounding signal (WSS).

U.S. patent application 20240126328 (von Badinski et al., Apr. 18, 2024, “Wearable Computing Device”) discloses a smart ring with a curved housing having a U-shape interior, a battery, a semi-flexible PCB, a galvanic sensor, light emitters, light receivers, a memory, a transceiver, a temperature sensor, and a processor. U.S. patent application 20240126329 (von Badinski et al., Apr. 18, 2024, “Wearable Computing Device”) discloses a wearable ring device with a ring-shaped housing, a printed circuit board, and a sensor module with infrared light emitters, visible light emitters, and light receivers. U.S. patent application 20240126330 (von Badinski et al., Apr. 18, 2024, “Wearable Computing Device”) discloses a wearable ring device with a ring-shaped housing comprising two metallic materials, a printed circuit board, light emitters, and light receivers.

U.S. patent application 20240126382 (Yoo, Apr. 18, 2024, “Wearable Device and Method for Controlling Same”) discloses a method of controlling a smart ring by sensing contact from a finger on an outer surface electrode on an outer circumference of the ring. U.S. patent application 20240134417 (von Badinski et al., Apr. 25, 2024, “Wearable Computing Device”) discloses a wearable ring device with a ring-shaped housing, a thermoelectric generator, a printed circuit board, light emitters, and light receivers. U.S. patent application 20240138721 (Eom et al., May 2, 2024, “Apparatus and Method for Estimating Concentration of Analyte Component”) discloses an apparatus for estimating a component level using a plurality of light sources with different central wavelengths and at least one light detector.

U.S. patent application 20240143027 (von Badinski et al., May 2, 2024, “Wearable Computing Device”) discloses a wearable ring device with a ring-shaped housing with one or more windows, a printed circuit board, light emitters, and light receivers. U.S. patent application 20240143028 (von Badinski et al., May 2, 2024, “Wearable Computing Device”) discloses a wearable ring device with a ring-shaped housing, a printed circuit board, and a sensor module that includes red light emitters, infrared light emitters, and light receivers. U.S. patent application 20240146350 (Gretarsson et al., May 2, 2024, “Smart Ring”) discloses a ring, band, or necklace with a pressure-sensitive mechanism that receives user input in the form of applied pressure.

U.S. Pat. No. 11,980,439 (Koskela et al., May 14, 2024, “Optical Sensor System of a Wearable Device, A Method for Controlling Operation of an Optical Sensor System and Corresponding Computer Program Product”) discloses a system comprising at least two photo transmitters, a photoreceiver, receiving electronics, and a microcontroller. U.S. Pat. No. 11,986,313 (Kinnunen et al., May 21, 2024, “Method and System for Monitoring and Improving Sleep Pattern of User”) discloses methods and systems for providing feedback to a user for adjusting their sleep pattern. U.S. patent application 20240168521 (von Badinski et al., May 23, 2024, “Wearable Computing Device”) discloses a wearable ring device with a ring-shaped housing, one or more temperature sensors, a printed circuit board, light emitters, and light receivers.

U.S. patent application 20240176425 (Wang et al., May 30, 2024, “Method for Controlling Wearable Device and Wearable Device”) discloses detecting an abnormal touch event on a display screen of a wearable device and enabling gesture recognition in response to the abnormal touch event. U.S. patent application 20240187407 (Mars et al., Jun. 6, 2024, “Methods and Apparatus for Facilitating NFC Transactions”) discloses a method for controlling a remote control device with a session token in response to an authentication request. U.S. Pat. No. 12,007,727 (Leith et al., Jun. 11, 2024, “Watch Band with Fit Detection”) discloses a watch band with an adjustable capacitor whose capacitance changes when the watch band configuration changes.

U.S. patent application 20240188834 (Kwon et al., Jun. 13, 2024, “Apparatus and Method for Measuring Blood Pressure”) discloses an apparatus for estimating blood pressure using a pulse wave sensor. U.S. patent application 20240188881 (Bonificio et al., Jun. 13, 2024, “Wearable Ring Device and Method of Monitoring Sleep Apnea Events”) discloses a finger-worn band with a pulse oximetry sensor on an inner surface of the band. U.S. Pat. No. 12,013,725 (von Badinski et al., Jun. 18, 2024, “Wearable Computing Device”) discloses a wearable ring device with a ring-shaped housing, a printed circuit board, a haptic feedback module, red light emitters, infrared light emitters, and light receivers.

U.S. patent application 20240201736 (von Badinski et al., Jun. 20, 2024, “Wearable Computing Device”) discloses a wearable ring device with a ring-shaped housing, a first conductive contact component, a second conductive contact component, a printed circuit board, light emitters, and light receivers. U.S. patent application 20240298966 (Ariza-Zambrano et al., Sep. 12, 2024, “Method and System for Detecting Food Intake Events from Wearable Devices and Non-Transitory Computer-Readable Storage Medium”) discloses a system and method for detecting food intake using wearable devices.

### SUMMARY OF THE INVENTION

A person's food consumption can be tracked automatically by a wearable device with a motion sensor and a camera which is worn on a person's finger or wrist. The camera is activated to record images when the person waves their hand back and forth over food. The resulting food images are then analyzed using artificial intelligence, machine learning, and/or pattern recognition to identify food types and estimate food quantities. In an example, the wearable device can be a finger ring or smart watch. In an example, the camera can be on a ventral portion of the device, with a focal vector which is substantially-parallel to the plane of the circumference of the wearable device and which points radially-outward from the circumferential center of the wearable device.

### BRIEF INTRODUCTION TO THE FIGURES

FIG. 1 shows oblique and close-up views of a finger ring with a motion sensor and a ventral camera for tracking food consumption.

FIG. 2 shows oblique and close-up views of a smart watch with a motion sensor and a ventral camera for tracking food consumption.

FIG. 3 shows two sequential views of a finger ring with a laser pointer scanning a meal on a plate.

FIG. 4 shows a system for tracking food consumption comprising a finger ring worn on a person's dominant arm and a smart watch worn on the person's non-dominant arm.

FIG. 5 shows a system for tracking food consumption comprising a finger ring and smart eyewear.

FIG. 6 shows how a finger ring with a ventral camera can record images of food from different angles as a person waves their hand over the food.

FIG. 7 shows a cross-sectional view of a finger ring for tracking food consumption with a motion sensor, ventral camera, ventral laser pointer, data processor, data transmitter, energy source, and gem/light.

FIG. 8 shows a cross-sectional view of a smart watch for tracking food consumption with a motion sensor, ventral camera, ventral laser pointer, data processor, data transmitter, energy source, and display screen.

FIG. 9 shows a cross-sectional view of a system for tracking food consumption wherein images recorded by a camera on a finger ring are displayed on a screen of a smart watch.

### DETAILED DESCRIPTION OF THE FIGURES

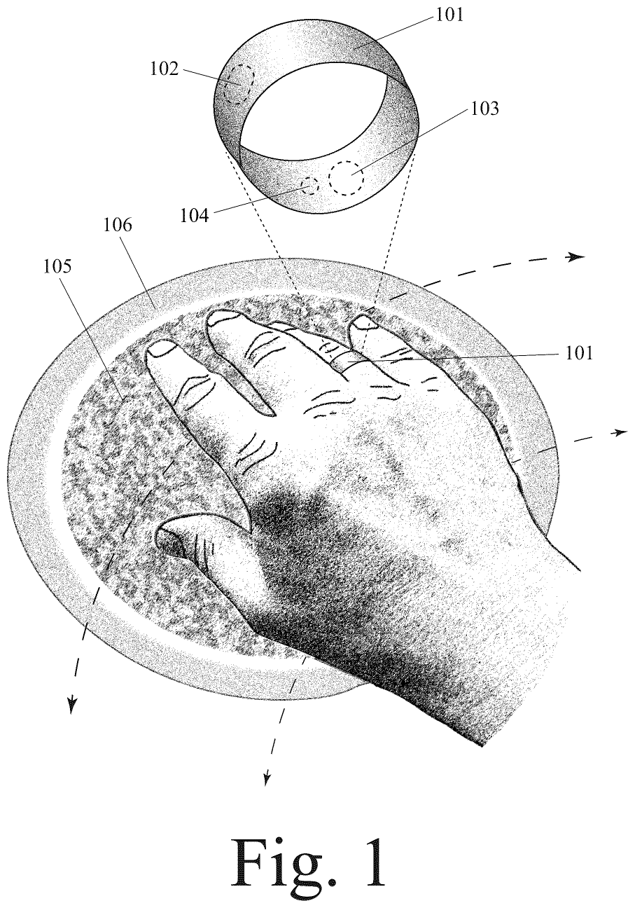

Before discussing the specific embodiments of this invention which are shown in FIGS. 1 through 9, this disclosure provides an introductory section which covers some of the general concepts, components, and methods which comprise this invention. Where relevant, these concepts, components, and methods can be applied as variations to the examples shown in FIGS. 1 through 9 which are discussed afterwards.

In the original Star Wars movies, there is a scene in which Obi-Wan Kenobi subtly waves his hand while planting thoughts in the mind of an imperial guard. During one hand wave he says—“These aren't the droids you are looking for.” This has become known as the “Jedi Mind Trick.” The Jedi Mind Trick is fictional. However, as disclosed herein, technology which enables a person to automatically track the types and quantities of food which they consume with just a subtle hand wave is feasible. The person just subtly waves their hand over food, which triggers a camera on the ventral side of a finger ring or smart watch to record food images which, in turn, are analyzed by machine learning, artificial intelligence, and/or pattern recognition. This technology enables food tracking with less social awkwardness than pulling out one's cellphone for each meal or leaning over food with camera-enabled smart glasses. If the person using the device wishes, they can also say under their breath--“These aren't the carbs you are looking for.” Disclosed herein is relatively-unobtrusive wearable technology for automated food consumption tracking based on subtle hand-waving motion.

In an example, a device for tracking a person's food consumption can comprise: a wearable device which is worn on a person's finger or wrist; a motion sensor on the wearable device; and a camera on a ventral portion of the wearable device; wherein data from the motion sensor is analyzed to detect a selected hand motion; wherein the camera is triggered to record images when the selected hand motion is detected; and wherein artificial intelligence, machine learning, and/or pattern recognition is used to analyze the images in order to identify types and quantities of food in the images.

In an example, the wearable device can be a finger ring. In an example, the wearable device can be a smart watch or other wrist-worn device. In an example, the camera can be removably-attached to a ventral portion of a watch band or strap. In an example, the camera can have a focal vector which is substantially-parallel to the plane of the circumference of the wearable device and which points radially-outward from the circumferential center of the wearable device. In an example, the selected hand motion can be a laterally-oscillating motion of the person's hand. In an example, the selected hand motion can be multiple back-and-forth cycles of laterally-oscillating motion of the person's hand.

In an example, the selected hand motion can be an eating-related hand-to-mouth motion. In an example, the selected hand motion can be multiple up-and-down cycles of eating-related hand-to-mouth motions. In an example, the selected hand motion can be a tap, swipe, or push on the device by the person's finger. In an example, the device can further comprise a laser pointer on the ventral portion of the device. In an example, the device can sequentially identify different portions and/or types of food in a multi-food meal based on sequential illumination of the different portions and/or types of food by the laser pointer.

In an example, a system for tracking food consumption can comprise: a finger ring worn on a finger of a person's dominant arm; wherein the finger ring further comprises a motion sensor, a data processor, a data transmitter, and an energy source; wherein the finger ring further comprises a camera on a ventral portion of the finger ring; and a smart watch or other wrist-worn device worn on a wrist of the person's non-dominant arm; wherein the smart watch or other wrist-worn device further comprises a display screen, wherein the camera records images of food, wherein the finger ring is in wireless communication with the smart watch or other wrist-worn device, and wherein images recorded by the camera are shown on the display.

In an example, the camera can have a focal vector which is substantially-parallel to the plane of the circumference of the finger ring and which points radially-outward from the circumferential center of the finger ring. In an example, the finger ring can further comprise a laser pointer on a ventral portion of the finger ring.

In an example, a method for tracking a person's food consumption can comprise: receiving motion data from a motion sensor on a wearable device which is worn on a person's wrist or finger; analyzing the motion data to detect a selected hand motion; triggering a camera on the wearable device to record images when the selected hand motion is detected; and using artificial intelligence, machine learning, and/or pattern recognition to analyze the images to identify types and quantities of food in the images.

In an example, the selected hand motion can be a laterally-oscillating motion of the person's hand. In an example, the selected hand motion can be multiple back-and-forth cycles of laterally-oscillating motion of the person's hand. In an example, the selected hand motion can be an eating-related hand-to-mouth motion. In an example, the selected hand motion can be a tap, swipe, or push on the device by the person's finger.

In an example, a motion sensor on a wearable device to track food consumption can comprise one or more of the following components: accelerometer, gyroscope, magnetometer, and inclinometer. In an example, a wearable device for tracking food consumption can receive commands via gesture recognition. In an example, a camera on a wrist-worn device can be a wide-angle camera.

In an example, a camera on a wearable device can be deactivated (e.g. stop recording images and/or power down) after a selected period of time in which no eating-related motions are detected. In an example, a camera on a wearable device can be triggered (e.g. activated) to start recording images of food on a dish when a selected hand motion is detected based on analysis of data from the motion sensor. In an example, a camera to record food images can be triggered to stop taking food images (e.g. deactivated) when data from a motion sensor indicates that it has been a selected (minimum) period of time since the last eating-related motion has been detected.

In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect a selected command gesture (e.g. tap, swipe, or shake) by the person's hand; triggering a camera on the device to record images when the selected command gesture is detected; and analyzing the images (e.g. using artificial intelligence, machine learning, and/or pattern recognition) to identify types and quantities of food in the images.

In an example, a wearable device for tracking food consumption can comprise: a finger ring which is worn on a person's finger; a motion sensor (e.g. accelerometer, gyroscope, magnetometer, and/or inclinometer) on the finger ring; and an outward-facing camera on the ventral side of the finger ring; wherein the camera is triggered to start recording images based on detection of a selected hand motion by analysis of data from the motion sensor.

In an example, a wearable device to measure food consumption can comprise a modular housing which can be attached to a ventral portion of a generic strap or band of a smart watch by a clamp, clasp, or clip; wherein the modular housing holds a motion sensor and a camera; and wherein the camera is triggered to start recording food images when analysis of data from the motion sensor detects a selected hand motion which is associated with eating. In an example, a wearable device to measure food consumption can include a push button, wherein pushing the button activates a camera on the device to record images. In an example, a finger ring for tracking food consumption can be worn on a person's ring finger.

In an example, a camera on a finger ring can be located on the ventral quadrant of the ring so that the camera captures images of food under the hand when the hand is waved with a palm-down orientation over food. In an example, a camera on the wrist-worn device (e.g. smart watch or watch band) can be located on a first lateral quadrant (e.g. between dorsal and ventral quadrants) and be oriented a ventral direction (e.g. have a ventral focal direction) which is substantially-tangential to the circumference of the device.

In an example, a wearable device for tracking food consumption can comprise: a finger ring which is worn on a person's finger; a camera on the ventral quadrant of the circumference of the finger ring; and a push button on the ventral quadrant of the circumference of the finger ring; wherein the camera is activated to start recording images when the button is pushed (e.g. by the person's thumb). In an example, a wearable device for tracking food consumption can include an LED on its dorsal quadrant, wherein the color and/or intensity of this light provides information concerning the accuracy (e.g. focal distance, angle, etc.) of food images being recorded, and wherein changes in the color and/or intensity help to guide the person concerning how to move their hand to improve the accuracy of food images being recorded.

In an example, food images recorded by a camera on a ventral quadrant of a finger ring can be shown on a display on the dorsal quadrant of the finger ring. In an example, a camera on a finger ring for recording food images can be located on a ventral location of the ring. In an example, a camera on a smart watch can be located on a side if a primary display housing of a smart watch. In an example, a camera to record food images can be located on the (circumferential) perimeter a smartwatch housing.

In an example, a camera to record food images can be in a modular housing is removably attached to a generic smart watch strap or band. In an example, a camera to record food images can be located on the band (or strap) of a smart watch. In an example, a finger ring for tracking food consumption can be in wireless electromagnetic communication with a wrist-worn device such as a smart watch, fitness band, or bracelet. In an example, a housing which contains a camera can be removably attached to a ventral portion of a smart watch or fitness band. In an example, a modular housing for a camera to record food images can have an opening, wherein modular housing is attached to the strap or band of a smart watch by sliding the strap or band through the opening.

In an example, a device can be part of a system for monitoring food consumption, wherein the system includes a wearable device on each of the person's arms and/or hands, wherein this system further comprises a wrist-worn device (e.g. smart watch) on the wrist of the person's non-dominant arm and a finger-worn device (e.g. finger ring) on a finger of the hand of the person's dominant arm, and wherein this system enables a person to wear the wrist-worn device on their non-dominant arm if this is their preference but still track use of their dominant arm for eating-related motions (e.g. hand-to-mouth motions). In an example, a device can be worn on the finger or wrist of a person's dominant arm in order to detect eating-related hand-to-mouth motions or gestures.

In an example, a camera on a finger ring can have a focal vector which is substantially-parallel to the plane of the finger ring and which points radially-outward from the circumferential center of the finger ring. In an example, a camera on a finger ring can have a focal vector which points downward when a person's hand is extended out horizontally with the palm facing downward. In an example, a camera on a wrist-worn device can have a focal vector which points downward when a person's hand is extended out flat (e.g. level with the ground) with the palm facing downward.

In an example, a wrist-worn device can include two cameras, wherein a first camera is on a first lateral side of the wrist (between the dorsal and ventral sides) and the second camera is on a second lateral side of the wrist (between the dorsal and ventral sides and opposite the first lateral side), wherein the focal vectors of both cameras are pointed in a ventral direction. In an example, the image in the field-of-view of the camera can be displayed to a person via a smart watch display with which the device is in wireless communication.

In an example, a camera on a finger ring can be triggered and/or activated to start recording images when a selected hand motion and/or gesture is detected based on analysis of data from a motion sensor, wherein this selected hand motion comprises the person moving their hand in a concave (e.g. semicircular) path in space with the palm of the hand oriented toward the food. In an example, a camera on a finger ring can be triggered and/or activated to start recording images when a selected hand motion and/or gesture is detected based on analysis of data from a motion sensor, wherein this selected hand motion is a laterally-oscillating hand motion (e.g. a hand wave) spanning between 6 and 18 inches in a substantially-horizontal plane with the palm of the hand facing downward.

In an example, a camera on a smart watch can be triggered to start recording images when data from a motion sensor in the smart watch indicates that the person wearing the smart watch is moving their hand back-and-forth, laterally, in a substantially-horizontal plane, with the palm of the hand facing downward. In an example, a camera on a ventral portion of a smart watch strap or band can be triggered to start recording images when data from a motion sensor in the smart watch indicates that the person wearing the smart watch is moving their hand back-and-forth, laterally, in a substantially-horizontal plane, with the palm of the hand facing downward.

In an example, a camera on the device can be activated (e.g. triggered) to record images of food by one or more mechanisms selected from the group consisting of: detection of at least one cycle of laterally-oscillating (e.g. back and forth) hand motion based on data from a motion sensor; detection of at least one hand-to-mouth gesture associated with eating based on data from a motion sensor; and detection of a selected tap, swipe, or touch on the device from the person wearing the device. In an example, a cycle of laterally-oscillating hand motion can comprise movement of a person's hand, palm facing downward, back and forth along an arcuate path in three-dimensional space over (a plate or bowl) of food.

In an example, a device for tracking a person's food consumption can comprise: a device (e.g. finger ring, wrist watch, watch band, or bracelet, other wrist-worn device) which is worn on a person's wrist or finger; a motion sensor (e.g. an accelerometer and gyroscope) on the device; and a camera on the device; wherein data from the motion sensor is analyzed to detect a cycle of laterally-oscillating motion (e.g. right-to-left and then left-to-right waving motion) by the person's hand, wherein the camera is triggered to record images when at least one cycle of laterally-oscillating hand motion is detected; and wherein the images are analyzed (e.g. using artificial intelligence, machine learning, and/or pattern recognition) to identify types and quantities of food in the images.

In an example, a laterally-oscillating hand motion can comprise a hand wave in a substantially-horizontal plane wherein the palm of the hand faces downward. In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect at least one cycle of laterally-oscillating motion (e.g. right-to-left and then left-to-right waving motion) by the person's hand; activating a camera on the device to record images each time that a cycle of laterally-oscillating hand motion is detected; and analyzing the images (e.g. using artificial intelligence, machine learning, and/or pattern recognition) to identify types and quantities of food in the images.

In an example, a person can rotate the palm of the hand as they move their hand along an arcuate path in three-dimensional space so that the focal vector of a camera on a finger ring remains pointed toward food. In an example, a selected hand motion can comprise at least one back-and-forth cycle of laterally-oscillating (e.g. right and left) hand motion with the palm of the hand facing generally-downward. In an example, a selected hand motion which triggers a camera to record food images can comprise movement of a hand in an arcuate path in three-dimensional space, wherein this arcuate path is concave, and wherein the food being imaged is located between 2 and 6 inches below the center of the concavity of this arcuate path.

In an example, a wearable device for tracking food consumption can be moved over and/or across food in an arcuate path in three-dimensional space as a person moves their hand, thereby recording images of the food from different angles. In an example, a wrist-worn device (e.g. smart watch) can be moved over food in an undulating and/or sinusoidal path in three-dimensional space so that a camera on a ventral portion of the device records images of the food from different angles to compile a 3D digital model of the food.

In an example, an arcuate path in three-dimensional space can be the path traced in three-dimensional space by movement of a finger ring on a person's hand. In an example, detection of a cycle of laterally-oscillating hand motion can be further specified to occur when a person extends their hand, palm downward, and then moves their hand from right-to-left and then left-to-right, or vice versa.

In an example, a camera on a wearable device can be triggered (e.g. activated) to start recording food images when analysis of data from a motion sensor (e.g. accelerometer, gyroscope, magnetometer, and/or inclinometer) detects repeated (e.g. a minimum number of) eating-related hand-to-mouth motions and/or gestures. In an example, a hand-to-mouth motion associated with eating can be identified based on a sequence of sub-motions, wherein this sequence of sub-motions can comprise: (a) upward movement of the hand, (b) rotating, tilting, or pivoting motion of the wrist, (c) a pause, and then (d) downward movement of the hand.

In an example, a wearable device for tracking food consumption can measure the speed, pace, or rate at which a person brings food up to their mouth (e.g. eating-related hand-to-mouth motions) and provide feedback to the person to encourage them to eat slower if the speed, pace, or rate is high. In an example, data from a motion sensor on a wearable device can be analyzed to estimate the rate of hand-to-mouth motions associated with eating. In an example, the number of hand-to-mouth motions detected by analysis of data from the motion sensor can be used in combination with analysis of food images to estimate the quantity of food consumed by the person.

In an example, a wearable device for tracking food consumption can track how fast a person is eating and provide feedback if the person is eating too fast. In an example, a device can display or speak, in a sequential manner, the type of food identified by the device for each portion food in a meal which comprises multiple types of food.

In an example, a device can emit a flashing and/or blinking light when analysis of data from a motion sensor indicates an eating-associated hand-to-mouth motion and/or gesture and the camera has not yet been activated to start recording images, wherein this flashing and/or blinking light prompts the person to activate the camera. In an example, a device can flash a light (e.g. LED display) when analysis of data from a motion sensor indicates a hand-to-mouth motion and/gesture which is associated with (e.g. probably indicative of) eating, wherein this flashing light prompts the person to wave their hand (back-and-forth) over food to record images of the food at a plurality of times during the course of a meal.

In an example, a device can periodically prompt a person to wave their hand over and/or across food at multiple times during a meal in order to record images of the food at different times during a meal, wherein changes in the volume of food over time are analyzed to estimate how much food a person has actually consumed during the meal. In an example, a person can be prompted to wave their hand over food to record food images after a selected number of eating-related hand-to-mouth motions has been detected by a wearable device. In an example, a wearable device for tracking food consumption can prompt a person to turn on a camera on the device and to wave their hand over food when a hand-to-mouth motion associated with eating is detected.

In an example, a wearable device for tracking food consumption can further comprise one or more computer-to-human interface components selected from the group consisting of: a display screen; a speaker or other sound-emitting member; a myostimulating member; a neurostimulating member; a speech or voice recognition interface; a synthesized voice; a vibrating or other tactile sensation creating member; MEMS actuator; an electromagnetic energy emitter; an infrared light projector; an LED or LED array; and an image projector.

In an example, a wearable device for tracking food consumption can have one or more vibrating components which provide haptic communication to the person wearing the device, wherein the device communicates different notifications, messages, and/or meanings via vibrations with different frequencies. In an example, a wearable device for tracking food consumption can have one or more vibrating components which provide haptic communication to the person wearing the device.

In an example, a wearable device to measure food consumption can vibrate when analysis of data from a motion sensor indicates a hand-to-mouth motion and/gesture which is associated with (e.g. probably indicative of) eating, wherein this vibration prompts the person to wave their hand back-and-forth over food to record images of the food (if they have not already done so). In another example, a wearable device for tracking food consumption can have one or more vibrating components which provide haptic communication to the person wearing the device, wherein the device communicates different notifications, messages, and/or meanings via different vibration durations.

In an example, a finger ring for tracking a person's food consumption can comprise: a finger ring worn by a person; wherein the finger ring further comprises a motion sensor, a data processor, a data transmitter, and an energy source (e.g. battery); wherein the finger ring further comprises a gem/light (e.g. a light emitting component with a gem-like or crystalline appearance) on a dorsal portion of the finger ring; wherein the finger ring further comprises a laser pointer (e.g. coherent light beam emitter) and a camera on a ventral portion of the finger ring; wherein the laser pointer directs a light beam onto food; and wherein the camera records images of the food.

In an example, a light emitter and/or projector on a device can emit one or more beams of coherent light which form a light pattern on a surface on which they shine, wherein this light pattern serves as a fiducial marker in analysis of food images to help measure food distance, orientation, size, and/or volume. In an example, a method for tracking a person's food consumption can comprise: tracking the movement of a (coherent) beam of light which sequentially shines on different types of food in a meal, wherein the beam of light is emitted from a device worn on a person's finger or wrist, and wherein the beam of light sequentially shines on different types of food in a meal as the person moves their hand over the meal.

In an example, a person can wave their hand over a multi-food meal so as to sequentially direct a light beam emitted from the device onto different types of food in the meal, wherein the person sequentially speaks to identify of each type of food (from their perspective) as the light beam shines on each type of food. In an example, the person's voice is received by a microphone on the device and the person's identifications of different food types are an input into the identification of food types and quantities by the device. In an example, a wearable device for tracking food consumption can further comprise a laser pointer which helps a person to move their hand to ensure that food is captured in images recorded by the camera.

In an example, a wearable device to measure food consumption can include one or more LEDs which emit one or more coherent light beams from the ventral quadrant of the device, wherein these one or more light beams form a pattern (e.g. a circle, a polygon, an array of dots, or an array of lines) on the surface which they hit, and wherein this pattern serves as a fiducial marker in recorded images to improve estimation of food distance, volume, shape, and/or size. In an example, a wearable device to measure food consumption can project a beam of (coherent) light, wherein this beam is used by the person to point the focal vector of a camera on the device sequentially toward different types of food in a meal.

In an example, analysis of food images can include identification of a food dish or utensil, wherein identification of a food dish or utensil helps to identify the type of food and/or the quantity of food. In an example, wearable device for tracking food consumption can further comprise a laser pointer, light emitter, and/or light projector which emits one or more beams of coherent light toward food, wherein these one or more beams form a geometric pattern (e.g. a circle, polygon, or matrix of dots) on or near the food. In an example, a camera on a wearable device can record an infrared light image of food. In an example, a wearable device for tracking food consumption can include an LED which illuminates food with visible light for recording images. In an example, a wearable device to measure food consumption can include a near-infrared light emitter.

In an example, a device for tracking a person's food consumption can comprise: a device (e.g. finger ring, wrist watch, watch band, or bracelet, other wrist-worn device) which is worn on the person's wrist or finger; a motion sensor (e.g. an accelerometer and gyroscope) on the device; a camera on the device; and spectroscopic sensor on the device; wherein data from the motion sensor is analyzed to detect a cycle of laterally-oscillating motion (e.g. right-to-left and then left-to-right waving motion) by the person's hand, wherein the camera is triggered to record images when at least one cycle of laterally-oscillating hand motion is detected, wherein the spectroscopic sensor is triggered to scan when at least one cycle of laterally-oscillating hand motion is detected, and wherein images recorded by the camera and spectroscopic data from the spectroscopic sensor are analyzed (e.g. using artificial intelligence, machine learning, and/or pattern recognition) to identify types and quantities of food in the images.

In an example, a wearable device for tracking food consumption can include one or more optical sensors selected from the group consisting of: spectroscopy sensor, spectrometry sensor, white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer. In an example, a wearable device to measure food consumption can include a spectroscopic sensor which measures changes in the spectral distribution of light caused by the interaction of light with the food.

In an example, a wearable device for tracking food consumption can be part of a system which further comprises a glucose sensor. In an example, a wearable device for tracking food consumption can further comprise one or more components selected from the group consisting of: display screen, electromagnetic energy sensor, gyroscope, humidity sensor, impedance sensor, microphone, power transducer, pressure sensor, proximity sensor, touch screen, and near-field communication (NFC) module.

In an example, a wearable device for tracking food consumption can have one or more human-to-computer communication interfaces through which the person can enter commands or other forms of communication to the device, wherein the one or more interfaces are selected from the group consisting of: camera; directing a low-power laser pointer; electromagnetic energy sensor; gesture recognition; motion recognition; motion sensor; muscle activation; optical sensor; pointing the device; pressing a button; pressure sensor; rotating a ring, dial, or crown; speaking to the device; tapping the device; touching or swiping a display screen; touching or swiping a portion of the circumference of the device; track ball; and voice recognition.

In an example, a wearable device to measure food consumption can include an electromyography (EMG) sensor. In an example, a finger ring can have a radially-asymmetric shape which helps to keep it from rotating around a person's finger, thereby keeping the camera on a ventral location. In an example, a finger ring for tracking food consumption can comprise: a dorsal half with a first width, wherein the width is measured in a plane which is tangential to the circumference of the device; and ventral half with a second width, wherein the width is measured in a plane which is tangential to the circumference of the device; wherein the ventral half is closer to the person's palm than the dorsal half; and wherein the first width is at least 25% greater than the second width.

In another example, a finger ring for tracking food consumption can comprise: a dorsal half with a first width, wherein the width is measured in a plane which is tangential to the circumference of the device; and ventral half with a second width, wherein the width is measured in a plane which is tangential to the circumference of the device; wherein the ventral half is closer to the person's palm than the dorsal half; and wherein the first width is between 100% and 200% greater than the second width. In an example, a finger ring for tracking food consumption can comprise: a first annular ring; a second annular ring, and a third annular ring; wherein the annular rings are coaxial and/or concentric, and wherein one or more of annular rings can be moved (e.g. rotated) relative to the other annular rings to control one or more functions of the device.

In an example, a finger ring for tracking food consumption can comprise: an inner annular member (e.g. ring) which is a first distance from the surface of a person's finger; and an outer annular member (e.g. ring) which is a second distance from the surface of the person's finger, wherein the second distance is greater than the first distance, wherein the outer annular member is rotated around the inner annular member, and wherein rotation of the outer annular member controls a device function selected from the group consisting of: consisting of: activation or change in focal direction of a camera on the device, activation or change in focal direction of a spectroscopic sensor on the device, change in the level and/or criteria for notifications conveyed to the person wearing the device, change in the level of power used by the device, change in the luminosity of a visual display on the device, change in the luminosity of an image projected by the device onto an external surface, change in the mode of computer-to-human communication interface (e.g. change between visual, auditory, and haptic communication) involving the device, change in the mode of human-to-computer communication interface (e.g. change between touch-based, voice command, and motion-based communication) involving the device, change in the volume of sound emitted from the device, change in the level of vibration created by the device, change in which biometric parameter is measured and/or displayed by the device, change in which other device is (or devices are) wirelessly-linked to the device, selecting a character or digit in a computer-based interaction, conveying the person's response to a notification, message, or call, and movement of a cursor on a different device.

In an example, a finger ring for tracking food consumption can be designed like a piece of jewelry. In an example, a finger ring for tracking food consumption can be in wireless electromagnetic communication with a hand-held device such as a cell phone. In an example, a preliminary list of food types identified in images can be presented to the person via a display on a cellphone with which the device is in wireless communication, providing an opportunity for the person to edit the list to correct errors. In an example, a wrist-worn or finger-worn device can be in wireless communication with smart eyewear, wherein camera images recorded by the wrist-worn or finger-worn device are displayed in the person's field-of-view by the display component of the smart eyewear. In an example, the image in the field-of-view of the camera can be displayed to a person via a cellphone with which the device is in wireless communication.

In an example, a device can be in wireless communication with another device (e.g. a different wearable device and/or a handheld device), wherein data from the motion sensor and/or images from the camera are analyzed in the other device. In an example, a wearable device for tracking food consumption can further comprise an energy transducer which converts light energy, thermal energy, or kinetic energy into electrical energy. In an example, a wearable device for tracking food consumption can be part of a system which further comprises a cardiac pacemaker. In an example, a wearable device for tracking food consumption can further comprise a Global Positioning System (GPS) component.

In an example, a wearable device for tracking food consumption can communicate food type identifications to the person wearing the device via an audio interface (e.g. computer-generated speech). In an example, communication and/or feedback from a wearable device to a person wearing the device can be sound-based, such as a tone or computer-generated voice. In an example, a finger ring for tracking food consumption can comprise: a camera on the ventral side of the finger ring; and a press button on the ventral side of the finger ring, wherein pressing the button turns the camera on.

In an example, a display on a wrist-worn device can show food images recorded by a camera located on a ventral portion of a finger ring. In an example, a camera on a wearable device can record video images of food as a person's hand is waved over the food, wherein these video images are integrated and/or compiled into a three-dimensional model of the food, and wherein this three-dimensional model of the food is used to measure food quantity. In an example, a wearable device to measure food consumption can house two cameras which in combination record stereoscopic images of food. In an example, images of food recorded from different angles and/or perspective can be integrated and/or combined into a three-dimensional image of the food which is then used to estimate food quantity.

In an example, a preliminary list of food types identified in images can be presented to the person via a display on the device and the person can be given an opportunity to edit the list to correct errors. In an example, a wearable device for tracking food consumption can provide a person with an opportunity to edit and/or correct automated food identifications, wherein the person can edit and/or correct these identifications by speaking into a microphone (e.g. on a wearable device for tracking food consumption or on a wirelessly-connected device). In an example, in the interest of privacy, images recorded by a camera on a finger ring can be processed to remove and/or blur images of people before those images are stored in digital memory.

In an example, data and/or images from a finger ring for tracking food consumption can be analyzed using one or more methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; kinematic modeling; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; machine learning; power spectral density; power spectrum analysis; and probit model.

In an example, a method for measuring a person's food consumption can comprise: receiving data from a motion sensor on a finger ring worn by a person; analyzing data from the motion sensor to detect a hand motion which is associated with eating; activating a camera on a ventral portion of the finger ring to record food images when a hand which is associated with eating is detected; and using machine learning and/or artificial intelligence to analyze the food images to identify food types and measure food quantities. In an example, food images recorded by a camera on a wearable device and/or data from a motion sensor on the wearable device can be analyzed using machine learning and/or artificial intelligence.

In an example, quantification of food quantities can be done by using machine learning and/or artificial intelligence to jointly analyze hand-to-mouth motions detected by a motion sensor and food images recorded by the camera. In an example, food images recorded by a camera on a wearable device and/or data from a motion sensor on the wearable device can be analyzed in order to estimate types and quantities of food. In an example, a wearable device for tracking food consumption can be part of a system with multiple motion sensors which recognizes hand motions and/or gestures. In an example, a wearable device for tracking food consumption which houses a motion sensor and a camera can be a finger ring.

In an example, a finger ring for tracking food consumption can have a camera which can be slid and/or rotated along an arcuate track on the ring. In an example, a camera on a wearable device can be triggered (e.g. activated) to start recording food images when analysis of data from a motion sensor (e.g. accelerometer, gyroscope, magnetometer, and/or inclinometer) detects a selected hand motion. In an example, a camera to record food images can be triggered to stop taking food images (e.g. deactivated) based on data from a motion sensor. In an example, a finger ring for tracking food consumption can have a partially-circumferential array of touch-activated buttons or switches.

In an example, a person can activate a camera on a wearable device to track food consumption by tapping, swiping, pressing, or otherwise touching the device. In an example, a wearable device for tracking food consumption can comprise: a wrist-worn device (e.g. smart watch) which is worn on a person's wrist; a motion sensor (e.g. accelerometer, gyroscope, magnetometer, and/or inclinometer) on the wrist-worn device; and an outward-facing camera on the ventral side of the wrist-worn device (e.g. on a ventral portion of a smart watch band or strap); wherein the camera is triggered to start recording images based on detection of a selected hand motion by analysis of data from the motion sensor.

In an example, a wearable device to measure food consumption can comprise a modular housing which can be attached to a ventral portion of a generic strap or band of a smart watch, wherein the modular housing holds a motion sensor and a camera, and wherein the camera is triggered to start recording food images when analysis of data from the motion sensor detects a selected hand motion which is associated with eating. In an example, a finger ring for tracking food consumption can be worn on a person's index finger. In an example, a wearable device for tracking food consumption which houses a motion sensor and a camera can be a wrist-worn device.

In an example, a camera on a finger ring for recording food images can be located on a ventral quadrant of the ring. In an example, a camera to record food images can be located on a ventral portion (e.g. ventral side or quadrant) of a wrist or arm bracelet. In an example, a wearable device for tracking food consumption can include a rotatable member on the ventral quadrant of a wearable device for tracking food consumption, wherein rotation of this member changes the focal direction of the camera, and wherein this member can be manually adjusted.

In an example, a wearable device for tracking food consumption can include an LED on its dorsal quadrant, wherein the color and/or intensity of this light provides information concerning the accuracy and/or usefulness (e.g. focal distance, angle, etc.) of food images being recorded. In an example, food images recorded by a camera on a ventral quadrant of a wrist-worn device can be shown on a display on the dorsal quadrant of the wrist-worn device.

In an example, a camera on a smart watch can be located on a primary display housing of a smart watch. In an example, a camera to record food images can be located in a smartwatch housing. In an example, a camera on a smart watch can be movably-attached to the band (or strap) of a wrist-worn device (e.g. smart watch) so that its radial location can be adjusted by sliding it along the circumference of the band (or strap). In an example, a camera to record food images can be located on a secondary housing of a smart watch, wherein the primary housing of the smart watch is located on a dorsal portion of the smart watch and the secondary housing is located on a ventral portion of the band (or strap) of the smart watch.

In an example, a device for tracking a person's food consumption can include a camera which is integrated into the band (or strap) of a smart watch. In an example, a housing which contains a camera can be attached to a band (or strap) of a smart watch strap by sliding the band (or strap) through an opening in the housing. In an example, a housing which contains a camera can be removably attached to a ventral portion of the band (or strap) of a smart watch. In an example, a wearable device for tracking food consumption which houses a motion sensor and a camera can be a smart watch, watch band, bracelet, or other wrist-worn device.

In an example, a device can be part of a system for monitoring food consumption, wherein the system includes a wearable device on each of the person's arms, such as a finger ring on one arm and a smart watch on the other arm. In an example, a system for measuring a person's food consumption can include a finger ring with a camera and motion sensor which is worn on a finger on the person's dominant arm and a wrist-worn device which is worn on the wrist of the person's non-dominant arm, wherein the finger ring and the wrist-worn device are in wireless communication with each other, and wherein images recorded by the camera are shown on a display on the wrist-worn device.

In an example, a camera on a finger ring can have a focal vector which points directly downward when a person's hand is extended out flat (e.g. level with the ground) with the palm facing downward. In an example, a camera on a wrist-worn device can have a focal vector which is substantially-parallel to the circumferential plane of the wrist-worn device and which points radially-outward from the circumferential center of the wrist-worn device. In an example, a finger ring for tracking food consumption can comprise an annular band which is worn around a person's finger and a camera on the annular band, wherein the focal direction of the camera is tangential to the circumference of the annular band. In an example, the focal direction and/or focal length of a camera on finger ring can be adjusted by the person wearing the ring by rotating a portion of the finger ring.

In an example, a camera on a finger ring can be triggered and/or activated to start recording images when a selected hand motion and/or gesture is detected based on analysis of data from a motion sensor, wherein this selected hand motion comprises the person moving their hand in an arcuate (e.g. conic section) path in space with the palm of the hand rotating during movement so as to remain substantially-oriented toward the food. In an example, a camera on a finger ring can be triggered and/or activated to start recording images when a selected hand motion and/or gesture is detected based on analysis of data from a motion sensor, wherein this selected hand motion comprises the person moving their hand in an arcuate (e.g. conic section) path in space with the palm of the hand oriented toward the food.

In an example, a camera on a finger ring can be triggered and/or activated to start recording images when a selected hand motion and/or gesture is detected based on analysis of data from a motion sensor, wherein this selected hand motion is a laterally-oscillating hand motion (e.g. a hand wave) spanning at least 6 inches in a substantially-horizontal plane with the palm of the hand facing downward. In an example, a camera on a smart watch strap or band can be triggered to start recording images when data from a motion sensor in the smart watch indicates that the person wearing the smart watch is moving their hand back-and-forth, laterally, in a substantially-horizontal plane, with the palm of the hand facing downward.

In an example, a camera on a wearable device can be triggered (e.g. activated) to start recording food images when analysis of data from a motion sensor (e.g. accelerometer, gyroscope, magnetometer, and/or inclinometer) detects a laterally-waving hand motion (with the palm of the hand facing generally downward). In an example, a cycle of laterally-oscillating (e.g. waving) hand motion can comprise a person moving (e.g. waving) their hand back-and-forth over food at a distance in the range between 3 inches and 2 feet from the food. In an example, a cycle of laterally-oscillating hand motion can comprise movement of a person's hand, palm facing downward, back and forth in an arc and/or curve over (a plate or bowl) of food.

In an example, a device for tracking a person's food consumption can comprise: a device (e.g. finger ring, wrist watch, watch band, or bracelet, other wrist-worn device) which is worn on the person's wrist or finger; a motion sensor (e.g. an accelerometer and gyroscope) on the device; a camera on the device; and a light emitter and/or projector on the device; wherein data from the motion sensor is analyzed to detect a cycle of laterally-oscillating motion (e.g. right-to-left and then left-to-right waving motion) by the person's hand, wherein the light emitter and/or projector is triggered to emit and/or project light when at least one cycle of laterally-oscillating hand motion is detected, wherein the camera is triggered to record images when at least one cycle of laterally-oscillating hand motion is detected, and wherein the images are analyzed (e.g. using artificial intelligence, machine learning, and/or pattern recognition) to identify types and quantities of food in the images.

In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect at least one cycle of laterally-oscillating motion (e.g. right-to-left and then left-to-right waving motion) by the person's hand; triggering a camera on the device to record images when at least one cycle of laterally-oscillating hand motion is detected; and analyzing the images (e.g. using artificial intelligence, machine learning, and/or pattern recognition) to identify types and quantities of food in the images.

In an example, a person can be guided concerning how to move their hand in an arcuate path in three-dimensional space over nearby food in order to capture food images from a plurality of angles and distances, wherein these food images from different angles and distances are integrated and/or combined to create a 3D model of the food. In an example, a person can wave their hand over and/or across food at multiple times during a meal in order to record images of the food at different times during a meal, wherein changes in the volume of food over time are analyzed to estimate how much food a person has actually consumed during the meal.

In an example, a selected hand motion which triggers a camera to record food images can comprise movement of a hand in an arcuate path in three-dimensional space, wherein the (plane of the) palm of the hand is rotated to remain substantially tangential to the curve of this arcuate path. In an example, a wearable device can guide a person (e.g. via audio or visual cues) concerning how to move their hand in an arcuate path in three-dimensional space over nearby food in order to capture food images from a plurality of angles and distances.

In an example, a wearable device to measure food consumption can include a speaker which emits sound, wherein the person is guided by sound cues (e.g. sound tones, levels, or volumes) concerning to move their hand in an arcuate path in three-dimensional space over nearby food in order to capture food images from a plurality of angles and distances, wherein these food images from different angles and distances are integrated and/or combined to create a 3D model of the food. In an example, an arcuate path in three-dimensional space along which a wearable device moves over food as a person moves their hand can be a conic section.

In an example, an arcuate path in three-dimensional space can be the path traced in three-dimensional space by movement of a particular location on a person's hand. In an example, detection of a cycle of laterally-oscillating hand motion can be further specified to occur when a person extends their hand, palm downward, out over (a plate or bowl of) food and then waves their hand over and/or across the food from right-to-left and then left-to-right, or vice versa. In an example, a camera to record food images can be triggered and/or activated to start recording images after a selected number of eating-related hand-to-mouth motions has been detected.

In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect at least one cycle of hand-to-mouth motion associated with eating; triggering a camera on the device to record images when at least one cycle of hand-to-mouth motion with eating is detected; and analyzing the images (e.g. using artificial intelligence, machine learning, and/or pattern recognition) to identify types and quantities of food in the images.

In an example, analysis of data concerning hand-to-mouth motions received from the motion sensor and analysis of food images can be combined to estimate food quantity. In an example, food type can be identified based on analysis of images recorded by a camera on a wearable device and food quantity can be estimated based on joint analysis of those images and the data from a motion sensor on the wearable device (e.g. number of eating-related hand-to-mouth motions detected). In an example, the quantity of food consumed by a person can be estimated based on a combination of: analysis of food images recorded by a camera on a wearable device; and analysis of motion data from a motion sensor on the wearable device (e.g. number of eating-related hand-to-mouth motions detected).

In an example, data from a motion sensor on a wearable device can be analyzed to estimate the speed and/or frequency with which a person brings food up to their mouth. In an example, a wearable device for tracking food consumption can be used to sequentially scan, identify, and quantify portions of different types of food in a multi-food meal. In an example, a device can flash a light (e.g. LED display) when analysis of data from a motion sensor indicates a hand-to-mouth motion and/gesture which is associated with (e.g. probably indicative of) eating, wherein this flashing light prompts the person to wave their hand back-and-forth over food to record images of the food at a plurality of times during the course of a meal to track decreases in the quantity of food left (and, by implication, how much the person has eaten).

In an example, a device can periodically (e.g. at regular time intervals) prompt a person to wave their hand over and/or across food at multiple times during a meal in order to record images of the food at different times during a meal, wherein changes in the volume of food over time are analyzed to estimate how much food a person has actually consumed during the meal. In an example, a device can prompt a person to wave their hand over and/or across food at multiple times during a meal in order to record images of the food at different times during a meal, wherein changes in the volume of food over time are analyzed to estimate how much food a person has actually consumed during the meal. In an example, a wearable device for tracking food consumption can prompt a person to continue scanning back and forth over food to record additional image data (e.g. images from additional angles and distances) until sufficient image data is recorded to create a satisfactory 3D model of the food.

In an example, a device can vibrate when analysis of data from a motion sensor indicates an eating-associated hand-to-mouth motion and/or gesture and the camera has not yet been activated to start recording images, wherein the vibration prompts the person to activate the camera. In an example, a wearable device for tracking food consumption can have one or more vibrating components which provide haptic communication to the person wearing the device, wherein different vibration durations and/or different lengths of inter-vibration pauses communicate different notifications, messages, and/or meanings to the person.

In an example, a wearable device for tracking food consumption can have one or more vibrating components which provide haptic communication to the person wearing the device, wherein the device communicates different notifications, messages, and/or meanings via different length pauses between vibrations. In an example, a wearable device for tracking food consumption can include piezoelectric actuators for communicating haptic (e.g. vibrational) notifications, messages, and/or signals to the person wearing the device.

In an example, a wearable device to measure food consumption can vibrate when analysis of data from a motion sensor indicates a hand-to-mouth motion and/gesture which is associated with (e.g. probably indicative of) eating, wherein this vibration prompts the person to wave their hand back-and-forth over food to record images of the food at a plurality of times during the course of a meal. In another example, a wearable device for tracking food consumption can have one or more vibrating components which provide haptic communication to the person wearing the device, wherein the device communicates different notifications, messages, and/or meanings via different vibration patterns with different increasing or decreasing durations.

In an example, a finger ring for tracking a person's food consumption can further comprise a light emitter (e.g. laser pointer) on the ventral side of the finger ring, wherein the light emitter emits light a beam of coherent toward the food. In an example, a light emitter and/or projector on a device can emit one or more beams of coherent light which form a light pattern on a surface on which they shine, wherein this light pattern serves as a fiducial marker. In an example, a person can move (e.g. scan) a finger ring over a meal, thereby shining a light beam emitted by the ring sequentially onto different types and/or portions of food in the meal.

In an example, a system for tracking a person's food consumption can comprise: a finger ring worn on a finger of a person's dominant arm (e.g. right arm); wherein the finger ring further comprises a motion sensor, a data processor, a data transmitter, an energy source (e.g. battery); wherein the finger ring further comprises a laser pointer (e.g. coherent light beam emitter) and a camera on a ventral portion of the finger ring; and a wrist-worn device (e.g. smart watch) worn on a wrist of the person's non-dominant arm (e.g. left arm); wherein the wrist-worn device further comprises a display (e.g. digital display screen); wherein the laser pointer directs a light beam onto food; wherein the camera records images of the food; wherein the wrist-worn device is in wireless communication with the finger ring; and wherein images recorded by the camera are shown on the display.

In an example, a wearable device for tracking food consumption can sequentially identify each type of food in the meal as each type of food in the meal is highlighted by a moving light beam emitted by the device. In an example, a wearable device to measure food consumption can project a beam of (coherent) light, wherein this beam is used by the person to direct the focal vector of a camera on the device toward food. In an example, a wrist-worn device (e.g. smart watch) can further comprise a light emitter (e.g. laser pointer) on the ventral side of the wrist-worn device, wherein the light emitter emits a beam of coherent light toward the food.

In an example, wearable device for tracking food consumption can further comprise a laser pointer, light emitter, and/or light projector which emits one or more beams of coherent light toward food, wherein the one or more beams form a geometric pattern (e.g. a circle, polygon, or matrix of dots) of light on or near the food, and wherein this geometric pattern is used to measure and/or calibrate the distance and/or angle of food during analysis of food images. In an example, wearable device for tracking food consumption can further comprise a laser pointer, light emitter, and/or light projector which emits one or more beams of coherent light toward food, wherein these one or more beams form a geometric pattern (e.g. a circle, polygon, or matrix of dots) on or near the food, and wherein this geometric pattern acts as a fiducial marker for estimating food distance, size, volume, and/or quantity.

In an example, a wearable device for tracking food consumption can include an LED which illuminates food with infrared or near-infrared light for recording images. In an example, a wearable device for tracking food consumption can include an LED which illuminates food with visible light. In an example, a camera on the device can be a multi-spectral camera. In an example. a wearable device (e.g. smart ring or smart watch) can include one or more spectroscopic (e.g. spectral measurement) sensors which are selected from the group consisting of: white light spectroscopy sensor, infrared spectroscopy sensor, near-infrared spectroscopy sensor, ultraviolet spectroscopy sensor, ion mobility spectroscopic sensor, mass spectrometry sensor, backscattering spectrometry sensor, and spectrophotometer.

In an example, a wearable device to measure food consumption can further comprise a plurality of light emitters which emit light in different spectral ranges, including at least one infrared light emitter. In an example, a wearable device to measure food consumption can include a spectroscopic sensor which provides information on the molecular composition of food based on changes in the spectral distribution of light caused by the interaction of light with that food.

In an example, a wearable device for tracking food consumption can be part of a system which further comprises a heart rate sensor. In an example, a wearable device for tracking food consumption can further comprise one or more components selected from the group consisting of: kinetic or thermal energy transducer; microphone; speaker; spectroscopic or other optical sensor; keypad, button, and/or turn knob; and tactile-sensation-creating member. In an example, a wearable device to measure food consumption can further comprise a touch screen and/or touch sensor. In an example, in addition to providing information about the types and quantities of food that a person is consuming, a wearable device can also provide information and/or sensory feedback to modify the person's food consumption for improved health.

In an example, a finger ring for tracking food consumption can be an annular ring with a flat-tire-shaped cross-section, wherein the flat portion spans between 30% and 50% of the circumference of the cross-section of the ring. In an example, a finger ring for tracking food consumption can have an inner perimeter (which faces toward the surface of the person's finger) and an outer perimeter (which faces away from the surface of the person's finger), wherein the inner perimeter has a circular shape and the outer perimeter has an oval or elliptical shape. In another example, a finger ring for tracking food consumption can have a partially-arcuate and partially-straight circumferential perimeter.

In an example, a finger ring for tracking food consumption can comprise: a first annular ring; and a second annular ring; wherein the first and second annular rings are coaxial and/or concentric, and wherein the second annular ring can be moved (e.g. rotated) relative to the first annular ring to control one or more functions of the device. In an example, a finger ring for tracking food consumption can be designed like a conventional finger ring with a gemstone, wherein the gemstone lights up with different colors, light intensity levels, and/or flashing light patterns to convey different messages to the person wearing the device. In an example, a gem/light can be an LED. In an example, a finger ring for tracking food consumption can be in wireless electromagnetic communication with electronically-functional and/or augmented reality eyewear.

In an example, a system for tracking food consumption can comprise: a finger ring which is worn on a finger on a person's dominant arm (e.g. right arm); a motion sensor (e.g. accelerometer, gyroscope, magnetometer, and/or inclinometer) on the finger ring; an outward-facing camera on the ventral side of the finger ring; smart eyewear (e.g. augmented reality eyeglasses) which is worn on the wrist of the person's non-dominant arm (e.g. left arm); and a display (e.g. digital display screen) on the wrist-worn device, wherein the finger ring and the wrist-worn device are in wireless communication with each other, and wherein images recorded by the camera on the finger ring are displayed on the display screen on the wrist-worn device.

In an example, the focal direction and/or focal length of a camera on finger ring can be adjusted by the person wearing the ring by touching a cellphone which is in wireless communication with the finger ring. In an example, the image in the field-of-view of the camera can be displayed to a person via smart eyewear with which the device is in wireless communication. In an example, a wearable device to measure food consumption can further comprise one or more additional components selected from the group consisting of: data processor; battery; wireless data transmitter; and wireless data receiver.

In an example, a wearable device to track food consumption can harvest, transduce, or generate electrical energy from kinetic energy, thermal energy, biochemical energy, ambient light energy, and/or ambient electromagnetic energy. In an example, a wearable device for tracking food consumption can be part of a system which further comprises a drug pump. In an example, a wearable device for tracking food consumption can further comprise one or more human-to-computer interface components selected from the group consisting of: buttons, knobs, dials, or keys; display screen; gesture-recognition interface; microphone; physical keypad or keyboard; virtual keypad or keyboard; speech or voice recognition interface; touch screen; EMG-recognition interface; and EEG-recognition interface.

In an example, a wearable device to measure food consumption can further comprise a microphone or other voice input device. In an example, a finger ring for tracking food consumption can include a plurality of LEDs which emit light, wherein emission of different light patterns communicates different notifications, messages, and/or signals to the person wearing the ring. In an example, a wearable device to measure food consumption can include a push button on a dorsal portion of the device. In an example, a display on a wrist-worn device can show food images recorded by a camera located on a ventral portion of the wrist-worn device.

In an example, a camera on a wearable device can record video images of food as a person's hand is waved over the food, wherein these video images are integrated and/or compiled into a three-dimensional model of the food. In an example, a wearable device to measure food consumption can include two cameras which capture images of food from different angles. In an example, a device can be calibrated by providing an opportunity the person wearing it to correct results (e.g. food identifications and quantity estimations), at least during a calibration period.

In an example, a preliminary list of food types identified in images can be presented to the person via text message, providing an opportunity for the person to edit the list to correct errors. In an example, a wearable device for tracking food consumption can provide a person with an opportunity to edit and/or correct automated food identifications, wherein the person can edit and/or correct these identifications by touching a touchscreen (e.g. on a wearable device for tracking food consumption or on a wirelessly-connected device). In an example, in the interest of privacy, images recorded by a camera on a finger ring can be processed to remove and/or blur images of people before those images are transmitted to an external device.

In an example, data and/or images from a finger ring for tracking food consumption can be analyzed using one or more methods selected from the group consisting of: multivariate linear regression or least squares estimation; factor analysis; Fourier Transformation; mean; median; multivariate logit; principal components analysis; spline function; auto-regression; centroid analysis; correlation; covariance; decision tree analysis; Kalman filter; linear discriminant analysis; linear transform; logarithmic function; logit analysis; Markov model; multivariate parametric classifiers; non-linear programming; orthogonal transformation; pattern recognition; random forest analysis; spectroscopic analysis; variance; artificial neural network; Bayesian filter or other Bayesian statistical method; chi-squared; eigenvalue decomposition; logit model; machine learning; power spectral density; power spectrum analysis; probit model; and time-series analysis.

In an example, data and/or images from a finger ring for tracking food consumption can be analyzed using machine learning and/or artificial intelligence. In an example, identification of food types can be done by using machine learning and/or artificial intelligence to analyze images recorded by the camera. In an example, the identification of food types and quantities consumed by the person can become part of a system for nutritional modification, improvement, and/or planning. In an example, a wearable device for tracking food consumption can include a set of motion sensors (e.g. accelerometers, gyroscopes, and/or magnetometers) which collects body motion data which is analyzed to detect when the person is eating food. In an example, a camera on a finger ring for recording food images can be a wide-angle camera.

In an example, a camera on a finger ring can be triggered and/or activated to start recording images when a selected hand motion and/or gesture is detected based on analysis of data from a motion sensor. In an example, a camera on a wearable device can be triggered (e.g. activated) to start recording food images when analysis of data from a motion sensor (e.g. accelerometer, gyroscope, magnetometer, and/or inclinometer) detects a selected hand motion. In an example, a camera to record food images can be triggered to stop taking food images (e.g. deactivated) when data from a motion sensor indicates that a meal is over.

In an example, a method for tracking a person's food consumption can comprise: detecting a selected type of tap or touch from a person's finger on a device (e.g. finger ring, wrist watch, watch band, or bracelet, other wrist-worn device) which is worn on the person's wrist or finger; triggering a camera on the device to record images; and analyzing the images (e.g. using artificial intelligence, machine learning, and/or pattern recognition) to identify types and quantities of food in the images. In an example, a wearable device for tracking food consumption can comprise: a finger ring which is worn on a person's finger; a camera on the ventral surface of the finger ring; and a push button on the ventral surface of the finger ring; wherein the camera is activated to start recording images when the button is pushed (e.g. by the person's thumb).

In an example, a wearable device for tracking food consumption can include a set of motion sensors (e.g. accelerometers, gyroscopes, and/or magnetometers) which collects body motion data which is analyzed to detect when the person is eating food, wherein detection of eating triggers a camera to start recording images. In an example, a wearable device to measure food consumption can comprise a modular housing which can be attached to a ventral portion of a generic strap or band of a smart watch, wherein there is an opening in the modular housing through which the strap or band is inserted, wherein the modular housing holds a motion sensor and a camera, and wherein the camera is triggered to start recording food images when analysis of data from the motion sensor detects a selected hand motion which is associated with eating.

In an example, a finger ring for tracking food consumption can be worn on a person's middle finger. In an example, wrist-worn device for tracking food consumption can be a bracelet. In an example, a camera on a wrist-worn device (e.g. smart watch or watch band) can be on the ventral quadrant of the device so that when the wrist is moved over and/or across food in a dorsal-up, ventral-down, orientation, the camera captures images of food under the wrist. In an example, a finger ring can be conceptually divided into four quadrants: a dorsal quadrant; a ventral quadrant; a first lateral quadrant (between the dorsal and ventral quadrants); and a second lateral quadrant (opposite the first lateral quadrant).

In an example, a wearable device for tracking food consumption can include a rotatable member on the ventral quadrant of a wearable device for tracking food consumption, wherein rotation of this member changes the focal direction of the camera. In an example, a wrist-worn device can be conceptually divided into four quadrants: a dorsal quadrant; a ventral quadrant; a first lateral quadrant (between the dorsal and ventral quadrants); and a second lateral quadrant (opposite the first lateral quadrant).

In an example, there can be a first camera on the wrist-worn device on the first lateral quadrant with a ventral focal direction (e.g. which is substantially-tangential to the circumference of the device) and a second camera on the wrist-worn device on the second lateral quadrant with a ventral focal direction (e.g. which is substantially-tangential to the circumference of the device). In an example, a camera on a smart watch can be located on a secondary housing of a smart watch, wherein a primary housing of the smart watch is located on a dorsal portion of the smart watch and the secondary housing is located on a ventral portion of the smart watch (e.g. opposite the primary housing). In an example, a camera to record food images can be located on a bracelet.

In an example, a camera to record food images can be in a modular housing is removably attached to a generic smart watch strap or band by a clip, clasp, clamp, or hook. In an example, a camera to record food images can be located on a ventral portion of a band (or strap) of a smart watch. In an example, a device for tracking a person's food consumption can include a camera which is part of the band (or strap) of a smart watch. In an example, a housing which contains a camera can be clamped and/or clipped onto a ventral portion of smart watch band (or strap).

In an example, a method for measuring a person's food consumption can comprise: receiving data from a motion sensor on a smart watch worn by a person; analyzing data from the motion sensor to detect a hand motion which is associated with eating; activating a camera on a ventral portion of the strap or band of the smart watch to record food images when a hand which is associated with eating is detected; and using machine learning and/or artificial intelligence to analyze the food images to identify food types and measure food quantities.

In an example, a device can be part of a system for monitoring food consumption, wherein the system includes a wearable device on each of the person's arms and/or hands, and wherein this system further comprises a wrist-worn device (e.g. smart watch) on the wrist of the person's non-dominant arm and a finger-worn device (e.g. finger ring) on a finger of the hand of the person's dominant arm. In an example, a device can be part of a system for monitoring food consumption, wherein the system includes a wearable device on each of the person's arms.

In an example, a system for tracking food consumption can comprise: a finger ring which is worn on a finger on a person's dominant arm (e.g. right arm); a motion sensor (e.g. accelerometer, gyroscope, magnetometer, and/or inclinometer) on the finger ring; an outward-facing camera on the ventral side of the finger ring; a wrist-worn device (e.g. smart watch) which is worn on the wrist of the person's non-dominant arm (e.g. left arm); and a display screen (e.g. digital display screen) on the wrist-worn device, wherein the finger ring and the wrist-worn device are in wireless communication with each other, and wherein images recorded by the camera on the finger ring are displayed on the display on the wrist-worn device.

In an example, a camera on a finger ring can have a focal vector which points downward and forward when a person's hand is extended out flat (e.g. level with the ground) with the palm facing downward. In an example, a camera on a wrist-worn device can have a focal vector which points downward and forward when a person's hand is extended out horizontally, with the palm facing downward. In an example, a wearable device to measure food consumption can include two cameras, wherein the focal vectors of these two cameras converge at a distance of between 6 and 24 inches from the cameras. In an example, the focal direction and/or focal length of a camera on finger ring can be adjusted by the person wearing the ring by touching a wrist-worn device which is in wireless communication with the finger ring.

In an example, a camera on a finger ring can be triggered and/or activated to start recording images when a selected hand motion and/or gesture is detected based on analysis of data from a motion sensor, wherein this selected hand motion comprises the person moving their hand in a concave path in space with the palm of the hand oriented toward the food. In an example, a camera on a finger ring can be triggered and/or activated to start recording images when a selected hand motion and/or gesture is detected based on analysis of data from a motion sensor, wherein this selected hand motion is a laterally-oscillating hand motion (e.g. a hand wave) spanning between 3 and 9 inches in a substantially-horizontal plane with the palm of the hand facing downward.

In an example, a camera on a finger ring can be triggered and/or activated to start recording images when a selected hand motion and/or gesture is detected based on analysis of data from a motion sensor, wherein this selected hand motion is a laterally-oscillating hand motion (e.g. a hand wave) in a substantially-horizontal plane with the palm of the hand facing downward. In an example, a camera on a ventral portion of a finger ring can be triggered to start recording images when data from a motion sensor in the finger ring indicates that the person wearing the ring is moving their hand back-and-forth, laterally, in a substantially-horizontal plane, with the palm of the hand facing downward.

In an example, a camera on a wearable device on a person's hand can be triggered (e.g. activated) to start recording images of food on a dish when the camera is triggered and/or activated by the motion of the hand waving (back and forth) over the food. In an example, a cycle of laterally-oscillating hand motion can comprise (waving) motion of a person's hand with its palm facing downward. In an example, a cycle of laterally-oscillating hand motion can occur when a person waves their hand from right-to-left and then left-to-right, or vice versa, over and/or across nearby food. In an example, a finger ring for tracking a person's food consumption can be moved over food in an undulating and/or sinusoidal path in three-dimensional space so that a camera on a ventral portion of the ring records images of the food from different angles to compile a 3D digital model of the food.

In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect at least one cycle of laterally-oscillating motion (e.g. right-to-left and then left-to-right waving motion) by the person's hand; activating a camera on the device to record images when at least one cycle of laterally-oscillating hand motion is detected; and deactivating the camera on the device when a selected period of time has passed since the least cycle of laterally-oscillating hand motion has been detected; and analyzing the images (e.g. using artificial intelligence, machine learning, and/or pattern recognition) to identify types and quantities of food in the images.

In an example, a person can move their hand over and/or across food in an arcuate path in three-dimensional space so that a camera on a device worn on their hand records images of the food from different angles, perspectives, and/or distances. In an example, a selected hand motion can comprise a hand moving (e.g. waving back and forth) over food in an arcuate path in three-dimensional space. In an example, a selected hand motion which triggers a camera to record food images can comprise movement of a hand in an arcuate path in three-dimensional space, wherein this arcuate path is concave, and wherein the food being imaged is located below the center of the concavity of this arcuate path.

In an example, a wearable device can guide a person by sound cues concerning how to move their hand in an arcuate path in three-dimensional space over nearby food in order to capture food images from a plurality of angles and distances, wherein these food images from different angles and distances are integrated and/or combined to create a 3D model of the food. In an example, a wearable device to measure food consumption can include light emitter, wherein the person is guided by light cues (e.g. different light colors, light flashes, or light intensities) concerning to move their hand in an arcuate path in three-dimensional space over nearby food in order to capture food images from a plurality of angles and distances, wherein these food images from different angles and distances are integrated and/or combined to create a 3D model of the food.

In an example, an arcuate path in three-dimensional space along which a wearable device moves over food as a person moves their hand can be a section of a circle or an ellipse. In an example, an arcuate path in three-dimensional space can be the path traced in three-dimensional space by movement of the centroid of a person's hand. In an example, a camera on a wearable device can be triggered (e.g. activated) to start recording food images when analysis of data from a motion sensor (e.g. accelerometer, gyroscope, magnetometer, and/or inclinometer) detects an eating-related hand-to-mouth motion and/or gesture.

In an example, a hand-to-mouth motion associated with eating can be identified based on a sequence of sub-motions, wherein this sequence of sub-motions can comprise: (a) contraction of the elbow and upward movement of the hand, (b) rotating, tilting, or pivoting motion of the wrist in a first direction. (c) a pause, (d) rotating, tilting, or pivoting motion of the wrist in the opposite direction, and then (e) extension of the elbow and downward movement of the hand. In an example, a selected hand motion can comprise an eating-related hand-to-mouth motion and/or gesture. In an example, analysis of data concerning hand-to-mouth motions received from the motion sensor and analysis of food images can be combined to estimate the quantity of food consumed by a person.

Selecting a ventral portion of a finger ring or smart watch as the preferred location for a camera to record food images is not an arbitrary or a random design variation. The most natural and subtle hand posture for a person's hand for the person to wave it laterally over food is to have their palm facing generally downward. With this posture, a camera on a ventral portion of a finger ring or smart watch easily captures images of food below the hand. If the camera were alternatively located on a dorsal portion of a finger ring or smart watch, then the person would have to wave their hand with their palm facing upward, which is less natural (e.g. almost contorting) and less subtle. If a camera on a finger ring were alternatively located on a lateral portion (between the ventral and dorsal portions), then its line-of-sight would likely be blocked by other fingers. If a camera on a smart watch were alternatively located on a lateral portion of its band (between the ventral and dorsal portions), then the person would have to wave their hand with the palm being vertical, which is much less subtle. This is why the ventral portion of a finger ring or smart watch is the preferred location for a camera in this invention, not just some arbitrary design variation.

In an example, the number and/or timing of hand-to-mouth motions can be analyzed to help identify the type and/or quantity of food consumed. In an example, the speed, rate, and/or frequency of hand-to-mouth motions can be analyzed to help identify the type and/or quantity of food consumed. In an example, a device can display or speak, in a sequential manner, the type of food identified by the device for each portion food in a meal which comprises multiple types of food, thereby giving the person an opportunity to edit and/or correct type identification for each portion of food. In an example, a wearable device for tracking food consumption can sequentially communicate to the person the identification for each type of food in the meal, giving the person an opportunity to edit and/or correct each food type identification (at least in a device calibration or learning period).

In an example, a device can flash a light (e.g. LED display) when analysis of data from a motion sensor indicates a hand-to-mouth motion and/gesture which is associated with (e.g. probably indicative of) eating, wherein this flashing light prompts the person to wave their hand back-and-forth over food to record images of the food (if they have not already done so). In an example, a device can periodically (e.g. every five to ten minutes) prompt a person to wave their hand over and/or across food at multiple times during a meal in order to record images of the food at different times during a meal, wherein changes in the volume of food over time are analyzed to estimate how much food a person has actually consumed during the meal.

In an example, a method for tracking a person's food consumption can comprise: receiving motion data concerning movement of a person's hand from a motion sensor (e.g. an accelerometer and gyroscope) on a device (e.g. finger ring, wrist watch, watch band, or bracelet, other wrist-worn device) which is worn on the person's wrist or finger; analyzing the motion data to detect at least one cycle of hand-to-mouth motion associated with eating; prompting the person to initiate at least one cycle of laterally-oscillating motion (e.g. right-to-left and then left-to-right waving motion) by the person's hand when at least one cycle of hand-to-mouth motion associated with eating is detected; triggering a camera on the device to record images when at least one cycle of laterally-oscillating hand motion is detected; and analyzing the images (e.g. using artificial intelligence, machine learning, and/or pattern recognition) to identify types and quantities of food in the images.

In an example, a wearable device for tracking food consumption can prompt a person to continue scanning back and forth over food to record additional image data (e.g. images from additional angles and distances) until sufficient image data is recorded to create a 3D model of the food that is sufficiently accurate to identify food types and estimate food quantities.

In an example, a wearable device for tracking food consumption can communicate different notifications, messages, and/or meanings to a person wearing the device via different vibration patterns, wherein differences in vibration patterns are selected from the group consisting of: first-order differences in vibration amplitude, first-order differences in vibration duration, first-order differences in vibration frequency, first-order differences in inter-vibration intervals, second-order differences (e.g. differences in increasing or decreasing pattern) in vibration duration, second-order differences (e.g. differences in increasing or decreasing patterns) in vibration amplitude, second-order differences (e.g. differences in increasing or decreasing patterns) in inter-vibration intervals, and second-order differences (e.g. differences in increasing or decreasing patterns) in vibration frequency.

In an example, a wearable device for tracking food consumption can have one or more vibrating components which provide haptic communication to the person wearing the device, wherein the device communicates different notifications, messages, and/or meanings via different vibration amplitudes. In an example, a wearable device for tracking food consumption can have one or more vibrating components which provide haptic communication to the person wearing the device, wherein the device communicates information via vibration patterns and/or sequences with different vibration durations and/or inter-vibration intervals.

In an example, a wearable device to measure food consumption can include a vibrating component which vibrates on the person's skin, wherein the person is guided by vibrational cues (e.g. vibration intervals or intensities) concerning to move their hand in an arcuate path in three-dimensional space over nearby food in order to capture food images from a plurality of angles and distances, wherein these food images from different angles and distances are integrated and/or combined to create a 3D model of the food.

In an example, a wearable device to measure food consumption can vibrate when analysis of data from a motion sensor indicates a hand-to-mouth motion and/gesture which is associated with (e.g. probably indicative of) eating, wherein this vibration prompts the person to wave their hand back-and-forth over food to record images of the food at a plurality of times during the course of a meal to track decreases in the quantity of food left (and, by implication, how much the person has eaten).

In an example, a device can display or speak, in a sequential manner, the type of food identified by the device for each portion food in a meal which comprises multiple types of food, thereby giving the person an opportunity to edit and/or correct type identification for each portion of food, wherein the sequence of food portions identified is determined by sequential illumination of the food portions by a light beam (e.g. laser pointer beam) emitted from the device. In an example, a light emitter and/or projector on a device can emit one or more beams of coherent light which form a light pattern on a surface on which they shine, wherein the person can adjust the location and/or orientation of their hand to ensure that nearby food is captured in images recorded by the camera.

In an example, a method for tracking a person's food consumption can comprise: analyzing data from a motion sensor in a device worn on a person's finger or wrist to detect at least one cycle of laterally-oscillating hand motions (e.g. hand wave back and forth with palm facing down); triggering a camera on the ventral side of the device to start recording images when at least one cycle of laterally-oscillating hand motions is detected; sequentially identifying types of food in a meal with portions of different types of food based on the recorded images, wherein sequential selection of food portions in the meal for analysis is based on sequential illumination of the food portions by a light beam emitted from the device; and wherein food identification is done using machine learning and/or artificial intelligence; and sequentially presenting the identifying types of food to the person, thereby enabling the person to edit and/or correct the identification of each type of food.

In an example, a person can move their hand to sequentially direct a light beam emitted from the device onto different components of a meal. In an example, a wearable device for tracking food consumption can comprise: a finger ring; a motion sensor (e.g. accelerometer, gyroscope, magnetometer, and/or inclinometer) on the finger ring; an outward-facing camera on the ventral side of the finger ring; and a light emitter (e.g. laser pointer) on the ventral side of the finger ring. wherein the light emitter emits a beam of light toward the food.

In an example, a wearable device to measure food consumption can include one or more LEDs which emit one or more coherent light beams from the ventral quadrant of the device. wherein these one or more light beams form a pattern (e.g. a circle, a polygon, an array of dots, or an array of lines) on the surface which they hit. In an example, a wearable device to measure food consumption can project a beam of (coherent) light, wherein this beam is used by the person to point the focal vector of a camera on the device sequentially toward different types of food in a meal, and wherein the person can also speak the type of food into a microphone on the device as each type of food is sequentially highlighted by the beam of light.

In an example, a wrist-worn device (e.g. smart watch) for tracking a person's food consumption can comprise: a wrist-worn device (e.g. smart watch) worn by a person; wherein the wrist-worn device further comprises a motion sensor, a data processor, a data transmitter, an energy source (e.g. battery); wherein the wrist-worn device further comprises a display (e.g. digital display screen) on a dorsal portion of the wrist-worn device; wherein the wrist-worn device further comprises a laser pointer (e.g. coherent light beam emitter) and a camera on a ventral portion of the wrist-worn device; wherein the laser pointer directs a light beam onto food; and wherein the camera records images of the food.

In an example, wearable device for tracking food consumption can further comprise a laser pointer, light emitter, and/or light projector which emits one or more beams of coherent light toward food, wherein the person uses this geometric pattern to ensure that the food is within the field-of-view of the camera. In an example, a camera on a wearable device can record an infrared image of food for thermal analysis, wherein this infrared image is used as part of food type identification. In an example, a wearable device for tracking food consumption can include an LED which illuminates food with infrared or near-infrared light. In an example, a wearable device to measure food consumption can include a near-infrared camera.

In an example, a camera on the device can record images in at least two different spectral ranges, including visible light and infrared light. In an example, a wearable device for tracking food consumption can include a plurality of light emitters (e.g. LEDs) which emit light in one or more of the following spectral ranges: infrared, near infrared, visible and/or white, and ultraviolet. In an example, a wearable device to measure food consumption can further comprise a plurality of light emitters which emit light in different spectral ranges. In an example, a wearable device to measure food consumption can include a spectroscopic sensor, wherein the spectroscopic sensor includes a light emitter which emits light toward the food and a light receiver which receives that light after it has interacted with the food, and wherein changes in the spectral distribution of light caused by this interaction are analyzed to provide information on the molecular composition of the food.

In an example, a wearable device for tracking food consumption can further comprise a pressure sensor. In an example, a wearable device for tracking food consumption can further include one or more sensors selected from the group consisting of: sound sensor, smell sensor, blood pressure sensor, heart rate sensor, ECG sensor, EMG sensor, electrochemical sensor, gastric activity sensor, GPS sensor, location sensor, optical sensor, piezoelectric sensor, respiration sensor, strain gauge, electrogoniometer, temperature sensor, and pressure sensor.

In an example, a wearable device to measure food consumption can include a near-infrared sensor. In an example, a finger ring can have a radially-asymmetric shape which helps to keep it from rotating around a person's finger and thereby helps to keep the camera on a ventral location, wherein a dorsal portion of a finger ring is wider and/or flatter than the rest of the circumference of the ring.

In an example, a finger ring for tracking food consumption can comprise: a dorsal half of an annular ring, wherein the dorsal half has a first average width, wherein width is measured in a plane which is tangential to the circumference of the device; and a ventral half of an annular ring, wherein the ventral half has a second average width, wherein width is measured in a plane which is tangential to the circumference of the device; wherein the ventral half is closer to the person's palm than the dorsal half; and wherein the first average width is at between 100% and 200% greater than the second average width.

In an example, a first portion of the circumferential perimeter of a finger ring for tracking food consumption can be arcuate (e.g. partially-annular and/or section of a circle) and a second portion of the circumferential perimeter of the electronic device can be straight (e.g. flat), wherein the first portion spans between 60% and 80% of the perimeter and the second portion spans between 20% and 40% of the perimeter.

In an example, a finger ring for tracking food consumption can comprise: a first annular member (e.g. ring) which is worn around a person's finger; a second annular member (e.g. ring) which is worn around the person's finger; and a rotational connection between the first annular member and the second annular member, wherein the rotational connection enables the second annular member to be rotated relative to the first annular member even though they are connected, and wherein the first annular member and the second annular member are parallel to each other, wherein the first annular member and the second annular member are connected in a manner which allows the second annular member to be rotated relative to the first annular member, and wherein rotation of the second annular member controls a device function selected from the group consisting of: consisting of: activation or change in focal direction of a camera on the device, activation or change in focal direction of a spectroscopic sensor on the device, change in the level and/or criteria for notifications conveyed to the person wearing the device, change in the level of power used by the device, change in the luminosity of a visual display on the device, change in the luminosity of an image projected by the device onto an external surface, change in the mode of computer-to-human communication interface (e.g. change between visual, auditory, and haptic communication) involving the device, change in the mode of human-to-computer communication interface (e.g. change between touch-based, voice command, and motion-based communication) involving the device, change in the volume of sound emitted from the device, change in the level of vibration created by the device, change in which biometric parameter is measured and/or displayed by the device, change in which other device is (or devices are) wirelessly-linked to the device, selecting a character or digit in a computer-based interaction, conveying the person's response to a notification. message, or call, and movement of a cursor on a different device.

In an example, a finger ring for tracking food consumption can comprise: an annular band which is worn around a person's finger; and a dorsal component (e.g. gem-like piece, dome, crown, or display screen) which protrudes radially-outward from the dorsal side of the annular band, wherein rotating the dorsal component controls a device function selected from the group consisting of: activation or change in focal direction of a camera on the device, activation or change in focal direction of a spectroscopic sensor on the device, change in the level and/or criteria for notifications conveyed to the person wearing the device, change in the level of power used by the device, change in the luminosity of a visual display on the device, change in the luminosity of an image projected by the device onto an external surface, change in the mode of computer-to-human communication interface (e.g. change between visual, auditory, and haptic communication) involving the device, change in the mode of human-to-computer communication interface (e.g. change between touch-based, voice command, and motion-based communication) involving the device, change in the volume of sound emitted from the device, change in the level of vibration created by the device, change in which biometric parameter is measured and/or displayed by the device, change in which other device is (or devices are) wirelessly-linked to the device, selecting a character or digit in a computer-based interaction, conveying the person's response to a notification, message, or call, and movement of a cursor on a different device.

In an example, a finger ring for tracking food consumption can be designed like a conventional finger ring with a gemstone. In an example, a gem/light can be configured to look like a light-emitting gemstone. In an example, a preliminary list of food types identified in images can be displayed to the person via smart eyewear, providing an opportunity for the person to edit the list to correct errors. In an example, a wearable device for tracking food consumption can communicate food type identifications to a person via a display on the device itself or on another device (such as smart phone, cell phone, or smart glasses) with which the device is in wireless communication. In an example, the function of a finger ring or wrist-worn device can be coordinated with the function of smart eyewear, wherein the locations of the device and the food can be tracked in the field-of-vision of the smart eyewear.

In an example, a power source for a wearable device to track food consumption can be: a power source that is internal to the ring during regular operation (such as an internal battery, capacitor, energy-storing microchip, wound coil or spring); a power source that is obtained, harvested, or transduced from a source other than a person's body that is external to the device (such as a rechargeable battery, electromagnetic inductance from external source, solar energy, indoor lighting energy, wired connection to an external power source, ambient or localized radiofrequency energy, or ambient thermal energy); or a power source that obtains, harvests, or transduces energy from a person's body (such as kinetic or mechanical energy from body motion, electromagnetic energy from a person's body, or thermal energy from a person's body).

In an example, an energy source for a wearable device can be a battery. In an example, a wearable device for tracking food consumption can be part of a system which further comprises an insulin pump. In an example, a finger ring for tracking a person's food consumption can further comprise an audio speaker. In an example, communication and/or feedback from a wearable device to a person wearing the device can be auditory feedback, such as a tone or a computer-generated voice.

In an example, a finger ring for tracking food consumption can comprise: a camera on the ventral side of the finger ring; a light emitter on the ventral side of the finger ring; and a press button on the ventral side of the finger ring, wherein pressing the button turns the camera and the light emitter on. In an example, a wearable device to measure food consumption can include a push button on a ventral portion of the device. In an example, a wrist-worn device (e.g. smart watch) can further comprise a display (e.g. digital display screen) on the dorsal side of the device.

In an example, a camera on a wearable device can record video images of food as a person's hand is waved over the food, wherein these video images can be integrated and/or compiled into a three-dimensional model of the food for better estimation of food volume and/or quantity. In an example, a wrist-worn device can include two cameras, wherein a first camera is on a first lateral side of the wrist (between the dorsal and ventral sides) and the second camera is on a second lateral side of the wrist (between the dorsal and ventral sides and opposite the first lateral side).

In an example, a device can enable a person to provide voice input concerning food identification and/or quantification, wherein this voice input supplements automated food identification and quantification based on images. In an example, a wearable device for tracking food consumption can give the person an opportunity to edit and/or correct each of preliminary automated identifications of food types and preliminary automated estimates of food quantities. In an example, a wearable device for tracking food consumption can provide a person with an opportunity to edit and/or correct automated food identifications.

In an example, one or more of the following characteristics of food in food images can be analyzed in order to identify types of food and measure quantities of food: colors, textures, shapes, edges, and contrast. In an example, the quantity of food consumed by a person can be estimated based on a multivariate analysis of: food images recorded by a camera on a wearable device; and data from a motion sensor on the wearable device (e.g. number of eating-related hand-to-mouth motions detected). In an example, food images recorded by a camera on a wearable device and/or data from a motion sensor on the wearable device can be analyzed using a neural network. In an example, quantification of food quantities can be done by using machine learning and/or artificial intelligence to analyze hand-to-mouth motions detected by a motion sensor and to analyze food images recorded by the camera.

In an example, a device for tracking a person's food consumption can comprise: a wearable device which is worn on a person's finger or wrist; a motion sensor on the wearable device; and a camera on a ventral portion of the wearable device; wherein data from the motion sensor is analyzed to detect a selected hand motion; wherein the camera is triggered to record images when the selected hand motion is detected; and wherein artificial intelligence, machine learning, and/or pattern recognition is used to analyze the images in order to identify types and quantities of food in the images.

In an example, the wearable device can be a finger ring. In an example, the wearable device can be a smart watch or other wrist-worn device. In an example, the camera can be removably-attached to a ventral portion of a watch band or strap. In an example, the camera can have a focal vector which is substantially-parallel to the plane of the circumference of the wearable device and which points radially-outward from the circumferential center of the wearable device. In an example, the selected hand motion can be a laterally-oscillating motion of the person's hand. In an example, the selected hand motion can be multiple back-and-forth cycles of laterally-oscillating motion of the person's hand.

In an example, the selected hand motion can be an eating-related hand-to-mouth motion. In an example, the selected hand motion can be multiple up-and-down cycles of eating-related hand-to-mouth motions. In an example, the selected hand motion can be a tap, swipe, or push on the device by the person's finger. In an example, the device can further comprise a laser pointer on the ventral portion of the device. In an example, the device can sequentially identify different portions and/or types of food in a multi-food meal based on sequential illumination of the different portions and/or types of food by the laser pointer.

In an example, a system for tracking food consumption can comprise: a finger ring worn on a finger of a person's dominant arm; wherein the finger ring further comprises a motion sensor, a data processor, a data transmitter, and an energy source; wherein the finger ring further comprises a camera on a ventral portion of the finger ring; and a smart watch or other wrist-worn device worn on a wrist of the person's non-dominant arm; wherein the smart watch or other wrist-worn device further comprises a display screen, wherein the camera records images of food, wherein the finger ring is in wireless communication with the smart watch or other wrist-worn device, and wherein images recorded by the camera are shown on the display.

In an example, the camera can have a focal vector which is substantially-parallel to the plane of the circumference of the finger ring and which points radially-outward from the circumferential center of the finger ring. In an example, the finger ring can further comprise a laser pointer on a ventral portion of the finger ring.

In an example, a method for tracking a person's food consumption can comprise: receiving motion data from a motion sensor on a wearable device which is worn on a person's wrist or finger; analyzing the motion data to detect a selected hand motion; triggering a camera on the wearable device to record images when the selected hand motion is detected; and using artificial intelligence, machine learning, and/or pattern recognition to analyze the images to identify types and quantities of food in the images.

In an example, the selected hand motion can be a laterally-oscillating motion of the person's hand. In an example, the selected hand motion can be multiple back-and-forth cycles of laterally-oscillating motion of the person's hand. In an example, the selected hand motion can be an eating-related hand-to-mouth motion. In an example, the selected hand motion can be a tap, swipe, or push on the device by the person's finger.

FIG. 1 shows an example of a wearable device for tracking food consumption comprising: a finger ring 101 which is worn on a person's finger; a motion sensor 102 on the finger ring; and an outward-facing camera 103 on the ventral side of the finger ring; wherein the camera is triggered to start recording images based on detection of a selected hand motion by analysis of data from the motion sensor. In an example, the selected hand motion can comprise at least one back-and-forth cycle of laterally-oscillating (e.g. right and left) hand motion with the palm of the hand facing generally-downward. In this example, the camera starts recording images of food 105 on a dish 106 when the camera is triggered (by the motion of the hand waving over the food). In this example, the finger ring further comprises a light emitter (e.g. laser pointer) 104 on the ventral side of the finger ring, wherein the light emitter emits light toward the food. The lower portion of FIG. 1 shows this finger ring being worn on a person's finger. The upper portion of FIG. 1 shows a close-up view of this finger ring alone. Relevant example variations discussed elsewhere in this disclosure or in priority-linked disclosures can also be applied to this example.

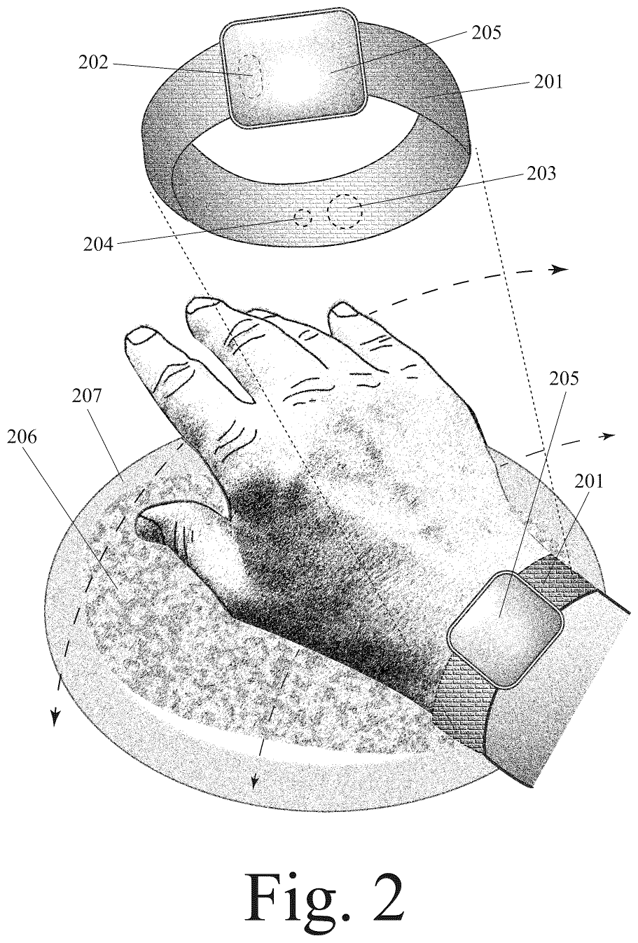

FIG. 2 shows an example of a wearable device for tracking food consumption comprising: a wrist-worn device (e.g. smart watch) 201 which is worn on a person's wrist; a motion sensor 202 on the wrist-worn device; and an outward-facing camera 203 on the ventral side of the wrist-worn device (e.g. a ventral portion of a strap for a smart watch); wherein the camera is triggered to start recording images based on detection of a selected hand motion by analysis of data from the motion sensor. In an example, the selected hand motion can comprise at least one back-and-forth cycle of laterally-oscillating (e.g. right and left) hand motion with the palm of the hand facing generally-downward. In this example, the camera starts recording images of food 206 on a dish 207 when the camera is triggered (by the motion of the hand waving over the food). In this example, the wrist-worn device further comprises a light emitter (e.g. laser pointer) 204 on the ventral side of the wrist-worn device, wherein the light emitter emits light toward the food. In this example, the wrist-worn device further comprises a display 205 (e.g. digital display screen) on the dorsal side of the device. The lower portion of FIG. 2 shows this wrist-worn device being worn on a person's wrist. The upper portion of FIG. 2 shows a close-up view of this wrist-worn device alone. Relevant example variations discussed elsewhere in this disclosure or in priority-linked disclosures can also be applied to this example.

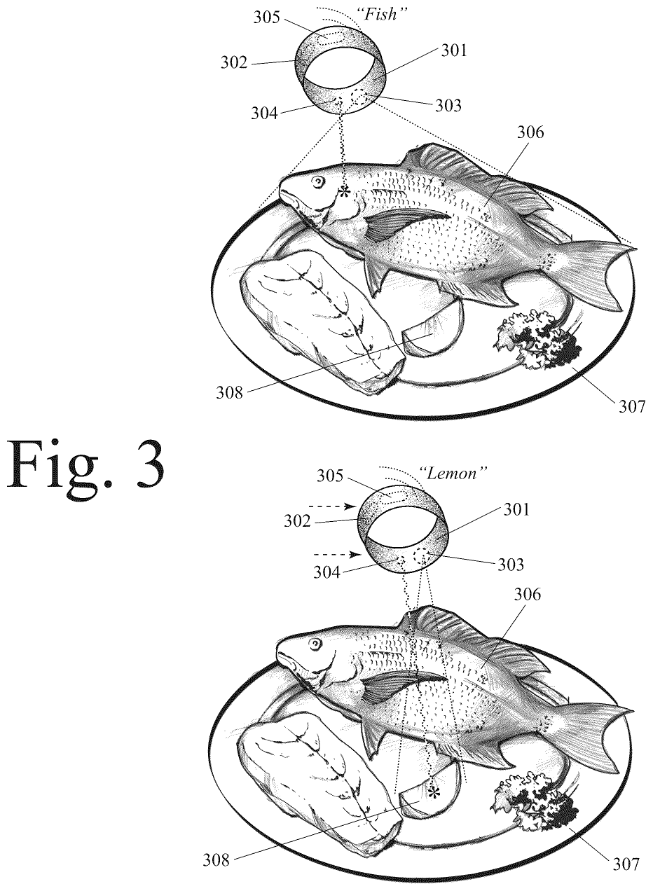

FIG. 3 shows an example of a wearable device for tracking food consumption being used to sequentially scan and identify portions of different types of food in a multi-food meal. The upper portion of FIG. 3 shows this device at a first time, when it is scanning fish in a meal. The lower portion of FIG. 3 shows this device at a second time when it is scanning a lemon (e.g. a lemon slice) in a meal. This wearable device for tracking food consumption comprises: a finger ring 301; a motion sensor 302 on the finger ring; an outward-facing camera 303 on the ventral side of the finger ring; and a light emitter (e.g. laser pointer) 304 on the ventral side of the finger ring, wherein the light emitter emits a beam of light toward the food. In this example, the finger ring further comprises a speaker 305. In an example, the person moves (e.g. scans) the finger ring over the food, shining the light beam sequentially onto different types of food in the meal. In an example, the device can sequentially identify each type of food in the meal as each type of food in the meal is highlighted by the light beam. In an example, identification of food types can be done by using machine learning and/or artificial intelligence to analyze images recorded by the camera. In an example, the device can sequentially communicate to the person the identification for each type of food in the meal. In an example, the device can give the person an opportunity to edit and/or correct each of these identifications. In this example, the device communicates food type identifications to the person via an audio interface (e.g. speech). In another example, a device can communicate food type identifications to a person via a display (on the device itself or another device such as a cell phone or smart glasses with which the device is in wireless communication). Relevant example variations discussed elsewhere in this disclosure or in priority-linked disclosures can also be applied to this example.

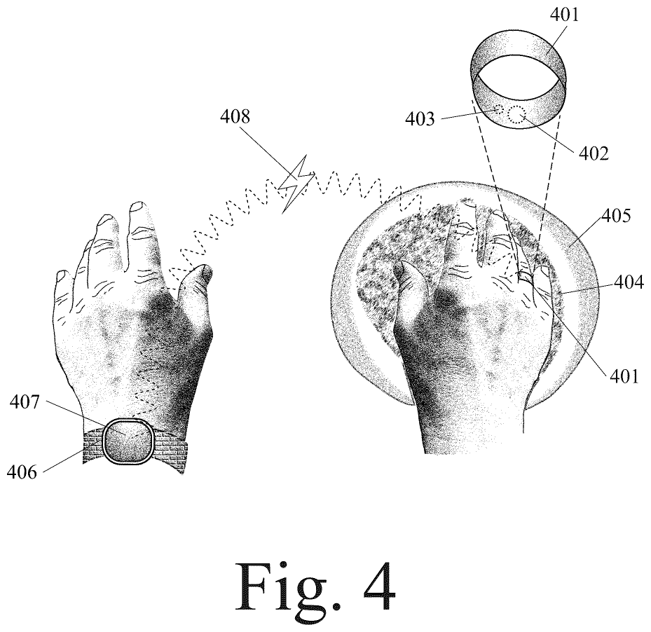

FIG. 4 shows an example of a system for tracking food consumption comprising: a finger ring 401 which is worn on a finger on a person's dominant arm; a motion sensor 403 on the finger ring; an outward-facing camera 402 on the ventral side of the finger ring; a wrist-worn device (e.g. smart watch) 406 which is worn on the wrist of the person's non-dominant arm; and a display screen 407 on the wrist-worn device, wherein the finger ring and the wrist-worn device are in wireless communication 408 with each other, and wherein images recorded by the camera on the finger ring are displayed on the display screen on the wrist-worn device. In an example, the camera can start recording images of food 404 on a dish 405 when a selected hand motion is detected based on analysis of data from the motion sensor. Relevant example variations discussed elsewhere in this disclosure or in priority-linked disclosures can also be applied to this example.

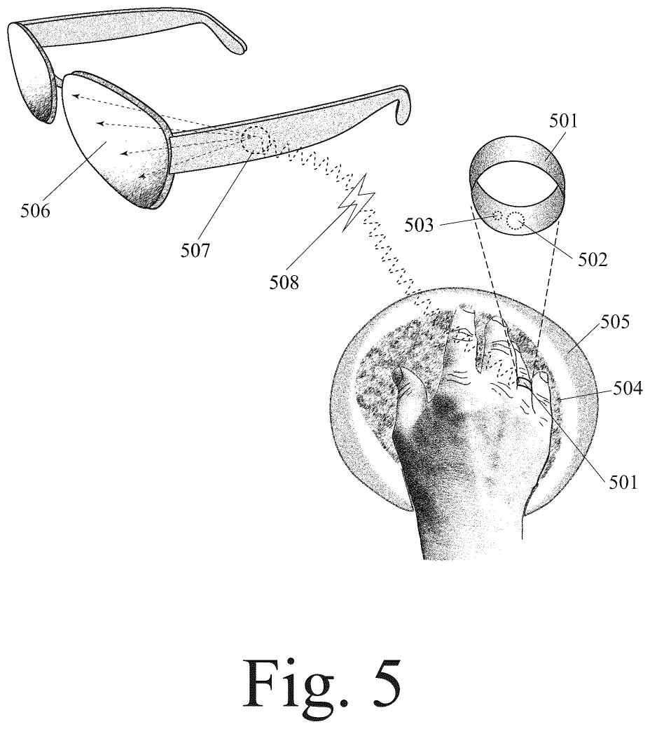

FIG. 5 shows an example of a system for tracking food consumption comprising: a finger ring 501 which is worn on a finger on a person's dominant arm; a motion sensor 503 on the finger ring; an outward-facing camera 502 on the ventral side of the finger ring; smart eyewear (e.g. augmented reality eyeglasses) 506 which is worn on the wrist of the person's non-dominant arm; and a display screen 507 on the wrist-worn device, wherein the finger ring and the wrist-worn device are in wireless communication 508 with each other, and wherein images recorded by the camera on the finger ring are displayed on the display screen on the wrist-worn device. In an example, the camera can start recording images of food 504 on a dish 505 when a selected hand motion is detected based on analysis of data from the motion sensor. Relevant example variations discussed elsewhere in this disclosure or in priority-linked disclosures can also be applied to this example.

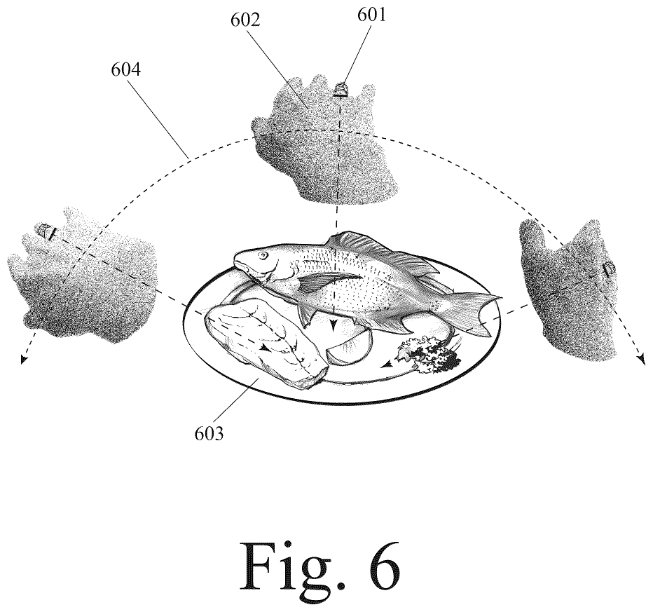

FIG. 6 shows an example of how a wearable device for tracking food consumption can be moved over and/or across food in an arcuate path in three-dimensional space, thereby recording images of the food from different angles. These images of food recorded from different angles can be integrated and/or combined into a three-dimensional image of the food which is then used to estimate food quantity. With respect to specific components, FIG. 6 shows a finger ring 601 worn on a person's hand 602, wherein the hand is moved over and/or across food 603 in an arcuate path 604 in three-dimensional space to record images of the food from different angles. In an example, the arcuate path in three-dimensional space can be the path traced in three-dimensional space by movement of a particular location on the hand. In an example, the arcuate path in three-dimensional space can be the path traced in three-dimensional space by movement of the centroid of the hand. In an example, the arcuate path in three-dimensional space can be the path traced in three-dimensional space by movement of the finger ring. In an example, the palm of the hand can be rotated as the hand moves along the arcuate path in three-dimensional space so that the focal vector of a camera on the finger ring remains pointed toward the food. In an example, the arcuate path can be a section of a circle or an ellipse. In an example, the arcuate path can be a conic section. Relevant example variations discussed elsewhere in this disclosure or in priority-linked disclosures can also be applied to this example.

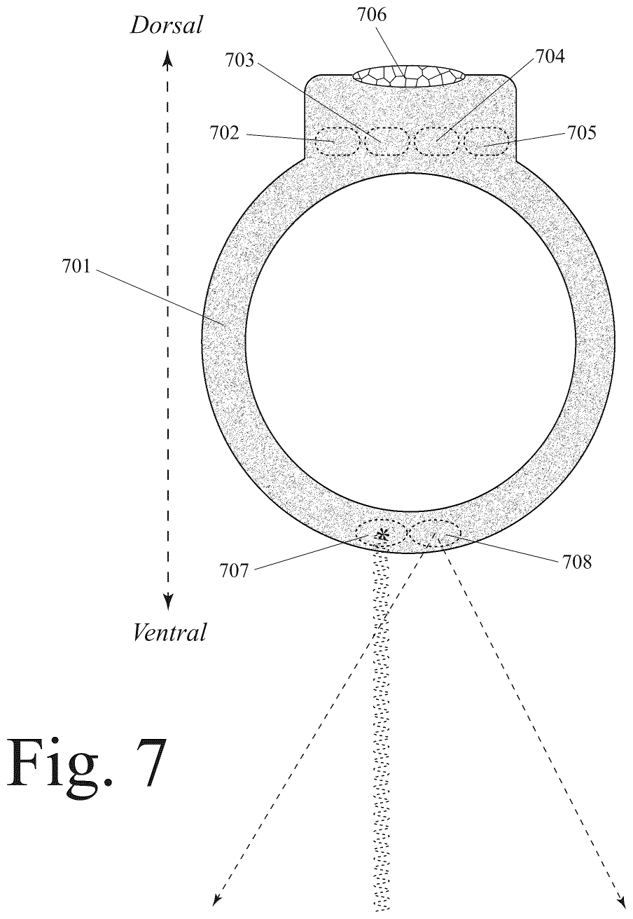

FIG. 7 shows a cross-sectional view of a finger ring for tracking a person's food consumption comprising: a finger ring 701 worn by a person; wherein the finger ring further comprises a motion sensor 702, a data processor 703, a data transmitter 704, and an energy source 705; wherein the finger ring further comprises a gem/light 706 on a dorsal portion of the finger ring; wherein the finger ring further comprises a laser pointer 707 and a camera 708 on a ventral portion of the finger ring; wherein the laser pointer directs a light beam onto food; and wherein the camera records images of the food. In an example, the camera can be triggered to start recording food images when analysis of data from the motion sensor detects a selected hand motion. In an example, the selected hand motion can be the hand moving (e.g. waving back and forth) over food in an arcuate path in three-dimensional space. In an example, the selected hand motion can be an eating-related hand-to-mouth motion. In an example, the gem/light can be an LED. In an example, the gem/light can be configured to look like a light-emitting gemstone. In an example, the energy source can be a battery. Relevant example variations discussed elsewhere in this disclosure or in priority-linked disclosures can also be applied to this example.

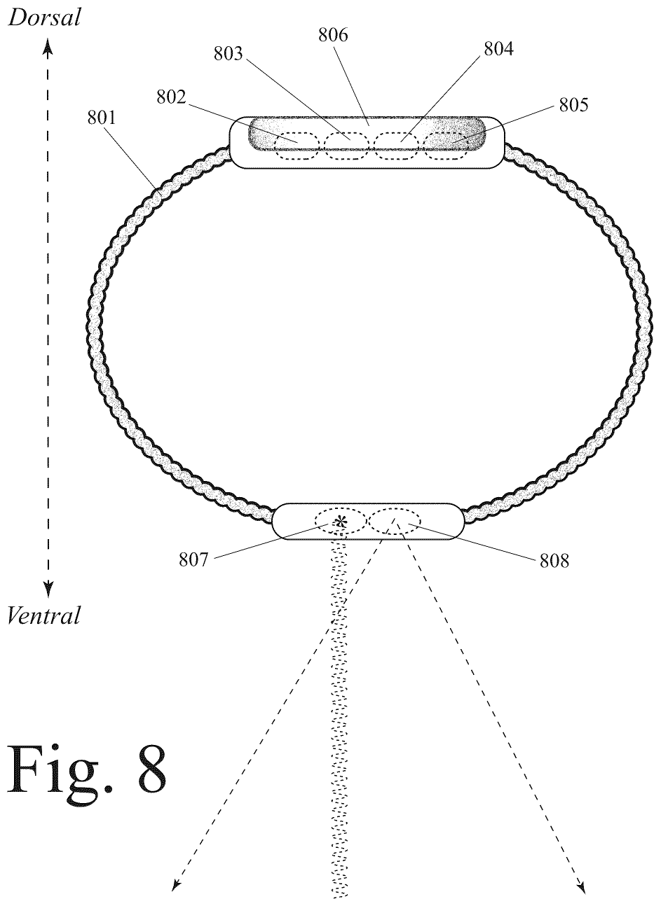

FIG. 8 shows a cross-sectional view of a wrist-worn device (e.g. smart watch) for tracking a person's food consumption comprising: a wrist-worn device (e.g. smart watch) 801 worn by a person; wherein the wrist-worn device further comprises a motion sensor 802, a data processor 803, a data transmitter 804, and an energy source 805; wherein the wrist-worn device further comprises a display screen 806 on a dorsal portion of the wrist-worn device; wherein the wrist-worn device further comprises a laser pointer 807 and a camera 808 on a ventral portion of the wrist-worn device; wherein the laser pointer directs a light beam onto food; and wherein the camera records images of the food. In an example, the camera can be triggered to start recording food images when analysis of data from the motion sensor detects a selected hand motion. In an example, the selected hand motion can be the hand moving (e.g. waving back and forth) over food in an arcuate path in three-dimensional space. In an example, the selected hand motion can be an eating-related hand-to-mouth motion. In an example, the display screen can show images recorded by the camera. In an example, the energy source can be a battery. Relevant example variations discussed elsewhere in this disclosure or in priority-linked disclosures can also be applied to this example.

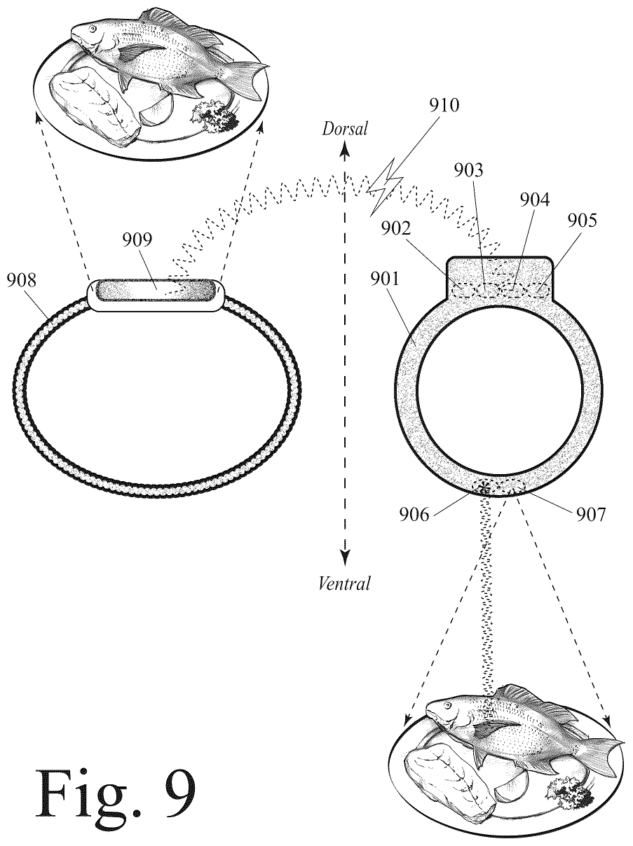

FIG. 9 shows a system for tracking a person's food consumption comprising: a finger ring 901 worn on a finger of a person's dominant arm; wherein the finger ring further comprises a motion sensor 902, a data processor 903, a data transmitter 904, and an energy source 905; wherein the finger ring further comprises a laser pointer 906 and a camera 907 on a ventral portion of the finger ring; and a wrist-worn device (e.g. smart watch) 908 worn on a wrist of the person's non-dominant arm; wherein the wrist-worn device further comprises a display (e.g. digital display screen) 909; wherein the laser pointer directs a light beam onto food; wherein the camera records images of the food; wherein the wrist-worn device is in wireless communication 910 with the finger ring; and wherein images recorded by the camera are shown on the display. In an example, the camera can be triggered to start recording food images when analysis of data from the motion sensor detects a selected hand motion. In an example, the selected hand motion can be the hand moving (e.g. waving back and forth) over food in an arcuate path in three-dimensional space. In an example, the selected hand motion can be an eating-related hand-to-mouth motion. In an example, the energy source can be a battery. Relevant example variations discussed elsewhere in this disclosure or in priority-linked disclosures can also be applied to this example.

I claim:

1. A device for tracking a person's food consumption comprising:
a wearable device which is worn on a person's finger or wrist;
a motion sensor on the wearable device; and
a camera on a ventral portion of the wearable device; wherein data from the motion sensor is analyzed to detect a selected hand motion; wherein the camera is triggered to record images when the selected hand motion is detected; and wherein artificial intelligence, machine learning, and/or pattern recognition is used to analyze the images in order to identify types and quantities of food in the images.

2. The device in claim 1 wherein the wearable device is a finger ring.

3. The device in claim 1 wherein the wearable device is a smart watch or other wrist-worn device.

4. The device in claim 3 wherein the camera is removably-attached to a ventral portion of a watch band or strap.

5. The device in claim 1 wherein the camera has a focal vector which is substantially-parallel to the plane of the circumference of the wearable device and which points radially-outward from the circumferential center of the wearable device.

6. The device in claim 1 wherein the selected hand motion is a laterally-oscillating motion of the person's hand.

7. The device in claim 1 wherein the selected hand motion is multiple back-and-forth cycles of laterally-oscillating motion of the person's hand.

8. The device in claim 1 wherein the selected hand motion is an eating-related hand-to-mouth motion.

9. The device in claim 1 wherein the selected hand motion is multiple up-and-down cycles of eating-related hand-to-mouth motions.

10. The device in claim 1 wherein the selected hand motion is a tap, swipe, or push on the device by the person's finger.

11. The device in claim 1 wherein the device further comprises a laser pointer on the ventral portion of the device.

12. The device in claim 11 wherein the device sequentially identifies different portions and/or types of food in a multi-food meal based on sequential illumination of the different portions and/or types of food by the laser pointer.

13. A system for tracking food consumption comprising:
a finger ring worn on a finger of a person's dominant arm;
wherein the finger ring further comprises a motion sensor, a data processor, a data transmitter, and an energy source;
wherein the finger ring further comprises a camera on a ventral portion of the finger ring; and
a smart watch or other wrist-worn device worn on a wrist of the person's non-dominant arm;
wherein the smart watch or other wrist-worn device further comprises a display screen, wherein the camera records images of food, wherein the finger ring is in wireless communication with the smart watch or other wrist-worn device, and wherein images recorded by the camera are shown on the display.

14. The device in claim 13 wherein the camera has a focal vector which is substantially-parallel to the plane of the circumference of the finger ring and which points radially-outward from the circumferential center of the finger ring.

15. The device in claim 13 wherein the finger ring further comprises a laser pointer on a ventral portion of the finger ring.

16. A method for tracking a person's food consumption comprising:
receiving motion data from a motion sensor on a wearable device which is worn on a person's wrist or finger;
analyzing the motion data to detect a selected hand motion;
triggering a camera on the wearable device to record images when the selected hand motion is detected; and
using artificial intelligence, machine learning, and/or pattern recognition to analyze the images to identify types and quantities of food in the images.

17. The method in claim 16 wherein the selected hand motion is a laterally-oscillating motion of the person's hand.

18. The method in claim 16 wherein the selected hand motion is multiple back-and-forth cycles of laterally-oscillating motion of the person's hand.

19. The method in claim 16 wherein the selected hand motion is an eating-related hand-to-mouth motion.

20. The method in claim 16 wherein the selected hand motion is a tap, swipe, or push on the device by the person's finger.
